(19) United States
(12) Patent Application Publication
(10) Pub. No.: US 2002/0128185 A1
(43) Pub. Date: Sep. 12, 2002

(54) Pharmaceutical compounds

(76) Inventor: Chuan Shih, Carmel, IN (US)

Correspondence Address: ELI LILLY AND COMPANY, LILLY CORPORATE CENTER, DROP CODE 1104, INDIANAPOLIS, IN, 46285, US

(21) Appl. No.: 09/990,895

(22) Filed: Nov. 16, 2001

Related U.S. Application Data
(63) Continuation-in-part of application No. 09/495,670, filed on Feb. 1, 2000.
Application No. 09/495,670 is a continuation-in-part of application No. 09/029,186, filed on Feb. 25, 1998, now abandoned.
Application No. 09/495,670 is a continuation-in-part of application No. 09/029,188, filed on Feb. 25, 1998, now abandoned.
Application No. 09/495,670 is a continuation-in-part of application No. 09/029,203, filed on Feb. 25, 1998, now abandoned.

(51) Int. Cl. A61K 38/12; C07K 5/12

(52) U.S. Cl. 514/9; 530/317; 540/454

(57) ABSTRACT

The invention provides novel cryptophycin compounds which can be useful for disrupting the microtubulin system, as antineoplastic agents, antifungal, and for the treatment of cancer. The invention further provides a formulation for administering the novel cryptophycin compounds.

This application is a continuation-in-part of U.S. application Ser. No. 09/495,670, filed Feb. 1, 2000, which is a continuation-in-part of U.S. application Ser. No. 09/029,186, U.S. application Ser. No. 09/029,188, and U.S. application Ser. No. 09/029,203, all of which were filed Feb. 25, 1998.

### FIELD OF THE INVENTION

This invention relates to the fields of pharmaceutical and organic chemistry and provides novel cryptophycin compounds useful as anti-microtubule agents.

### BACKGROUND OF THE INVENTION

Neoplastic diseases, characterized by the proliferation of cells not subject to the normal control of cell growth, are a major cause of death in humans and other mammals. Clinical experience in cancer chemotherapy has demonstrated that new and more effective drugs are desirable to treat these diseases. Such clinical experience has also demonstrated that drugs that disrupt the microtubule system of the cytoskeleton can be effective in inhibiting the proliferation of neoplastic cells. Further, such agents having the ability to disrupt the microtubule system can be useful for research purposes.

The microtubule system of eucaryotic cells is a major component of the cytoskeleton and is a dynamic assembly and disassembly. Thus heterodimers of tubulin are polymerized and form microtubule. Microtubules play a key role in the regulation of cell architecture, metabolism, and division. The dynamic state of microtubules is critical to their normal function. With respect to cell division, tubulin is polymerized into microtubles that form the mitotic spindle. The microtubules are then depolymerized when the mitotic spindle's use has been fulfilled. Accordingly, agents that disrupt the polymerization or depolymerization of microtubules, and thereby inhibit mitosis, comprise some of the most effective cancer chemotherapeutic agents in clinical use.

Such anti-mitotic agents or poisons may be classified into three groups on the basis of their molecular mechanism of action. The first group consists of agents, including colchicines and colcemid, that inhibit the formation of microtubules by sequestering tubulin. The second group consists of agents, including vinblastine and vincristine, which induce the formation of paracrystalline aggregates of tubulin. The third group consists of agents, including taxol, that promote the polymerization of tubulin and thus stabilize microtubules. All of these three groups are well known anti-cancer drugs: their action of disrupting mitotic spindle microtubules preferentially inhibits hyperproliferative cells.

The exhibition of drug resistance and multiple-drug resistance phenotype by many tumor cells and the clinically proven mode of action of anti-microtubule agents against neoplastic cells necessitates the development of anti-microtubule agents cytotoxic to non-drug resistant neoplastic cells as well as cytotoxic to neoplastic cells with a drug resistant phenotype.

Certain cryptophycin compounds are known in the literature; however, cryptophycin compounds having even greater solubility with robust potency are desired for most pharmaceutical uses and a broader library of cryptophycin compounds could provide additional treatment options. Applicants have now discovered novel compounds providing such desired solubility as well compounds having the ability to disrupt the microtubule system. Such compounds can be prepared using total synthetic methods and are therefore well suited for development as pharmaceutically useful agents.

### SUMMARY OF THE INVENTION

The presently claimed invention provides novel cryptophycin compounds of Formula I

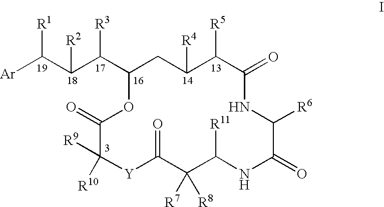

wherein

Ar is any simple unsubstituted aromatic group, simple substituted aromatic group, simple unsubstituted heteroaromatic group, simple substituted heteroaromatic group, C₁-C₁₂ alkyl, C₂-C₁₂ alkyne;

R¹ is halogen, SH, amino, monoalkylamino, dialkylamino, trialkylammonium, alkylthio, dialkylsulfonium, sulfate, or phosphate;

R² is hydroxy or SH; or

R¹ and R² may be taken together to form an epoxide ring, an aziridine ring, an episulfide ring, a sulfate ring, a cyclopropyl ring, or monoalkylphosphate ring; or

R¹ and R² may be taken together to form a second bond between C₁₈ and C₁₉;

R³ is a lower alkyl group;

R⁴ is hydrogen;

R⁵ is hydrogen;

R⁴ and R⁵ may be taken together to form a second bond between C₁₃ and C₁₄;

R⁶ is a substituent selected from the group consisting of unsubstituted B-ring heteroaromatic, substituted B-ring heteroaromatic, (C₃-C₈)cycloalkyl, substituted C₃-C₈ cycloalkyl, substituted (C₁-C₆)alkyl, unsubstituted (C₁-C₆)alkyl, a group of the formula III′:

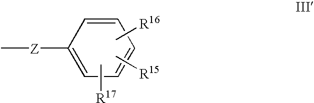

and a group of the formula III″:

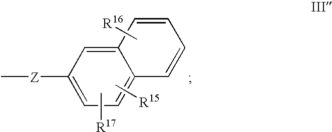

R⁷ is selected from the group consisting of NR⁵¹R⁵²,

R⁵³NR⁵¹R⁵², OR⁵³, hydrogen and a lower alkyl group; R⁵¹ and

R⁵² are independently selected from the group consisting of C₁-C₃ alkyl; R⁵³ is C₁-C₃ alkyl;

R⁸ is hydrogen or a lower alkyl group;

R⁷ and R⁸ can optionally form a spiro group;

R⁹ is selected from the group consisting of hydrogen, a lower alkyl group, unsaturated lower alkyl, and lower alkyl-C₃-C₅ cycloalkyl;

R¹⁰ is hydrogen or a lower alkyl group;

R⁹ and R¹⁰ together optionally form a cyclopropyl ring;

R¹¹is selected from the group consisting of hydrogen, hydroxy, simple alkyl, unsubstituted phenyl, substituted phenyl, unsubstituted benzyl, and substituted benzyl;

R¹⁵, R¹⁶, and R¹⁷ are each independently selected from the group consisting of hydrogen, (C₁-C₆)alkyl, OR¹⁸, halo, NR¹⁸′R¹⁹′, NO₂, OPO₄H₂, OR¹⁹phenyl, SCH₂phenyl, CONH₂, CO₂H, PO₃H₂, and SO₂R²³, and ZZ;

R¹⁸ is selected from the group consisting of hydrogen, aryl, and C₁-C₆ alkyl;

R¹⁸′ is selected from the group consisting of hydrogen and (C₁-C₆)alkyl;

R¹⁹ is C₁-C₆ alkyl;

R¹⁹′ is selected from the group consisting of hydrogen and (C₁-C₆)alkyl;

R²³ is selected from the group consisting of hydrogen and (C₁-C₃)alkyl;

n is 0, 1, or 2;

p is 0, 1, or 2;

m is 0, 1, or 2;

Y is selected from the group consisting of O and NH;

Z is selected from the group consisting of —(CH₂)ₙ—, —(CH₂)ₚ—O—(CH₂)ₘ— and (C₃-C₅)cycloalkyl;

ZZ is selected from the group consisting of a simple unsubstituted aromatic group and a simple substituted aromatic group; or a pharmaceutically acceptable salt or solvate thereof.

The present invention provides pharmaceutical formulations, a method for disrupting a microtubulin system using an effective amount of a compound of Formula I, a method for inhibiting the hyperproliferation of mammalian cells comprising administering an effective amount of a compound of Formula I, and a method for treating neoplasia in a mammal comprising administering an effective amount of a compound of Formula I.

### DETAILED DESCRIPTION OF THE INVENTION

As used herein, the term “lower alkyl” shall refer to (C₁-C₇)alkyl wherein the alkyl may be saturated, branched, or straight chain or a (C₂-C₇)alkyl wherein the alkyl may be unsaturated. Examples include, but are in no way limited to, methyl, ethyl, n-propyl, iso-propyl, n-butyl, propenyl, sec-butyl, n-pentyl, isobutyl, tert-butyl, sec-butyl, methylated butyl groups, pentyl, tert pentyl, sec-pentyl, methylated pentyl groups and the like.

As used herein, the term “substituted phenyl” shall refer to a phenyl group with from one to three which may be independently selected from the group consisting of lower alkyl, Cl, Br, F, and I.

As used herein, the term “substituted benzyl” shall refer to a benzyl group with from one to three substitutents which may be independently selected from the group consisting of simple alkyl, Cl, Br, F, and I wherein such substituents may be attached at any available carbon atom.

As used herein “unsubstituted B-ring heteroaromatic group” refers to aromatic rings which contain one or more non-carbon member selected from the group consisting of oxygen, nitrogen, and sulfur. As used herein “substituted B-ring heteroaromatic group” refers to aromatic rings that contain one or more non-carbon members selected from the group consisting of oxygen, nitrogen, and sulfur with substituents selected from the group consisting of OR²⁰. Especially preferred embodiments are selected from, but not limited to,

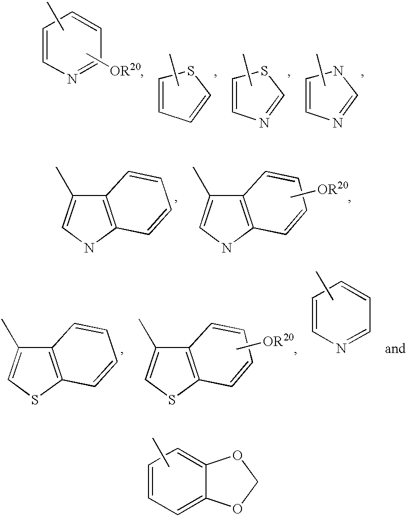

wherein R²⁰ is selected from hydrogen and C₁-C₆ alkyl.

It is especially preferred that “B-ring heteroaromatic group” refers to a substituent selected from the group consisting of:

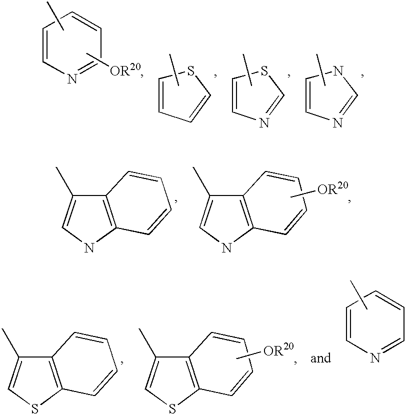

As used herein “cycloalkyl” refers to a saturated C₁-C₈ cycloalkyl group wherein such group may include from zero to three substituents selected from the group consisting of C₁-C₃ alkyl, halo, and OR²² wherein R²² is selected from hydrogen and C₁-C₃ alkyl. Such substituents may be attached at any available carbon atom. It is especially preferred that cycloalkyl refers to substituted or unsubstituted cyclohexyl.

As used herein, “spiro group” refers to C₃-C₈ cycloalkyl, preferably cyclopropyl, cyclobutyl, and cyclopentyl. The spiro group is most preferably cyclopropyl.

As used herein “lower alkoxyl group” means any alkyl group of one to five carbon atoms bonded to an oxygen atom. As used herein “lower alkyl group” means an alkyl group of one to five carbons and includes linear and non-linear hydrocarbon chains, including for example, but not limited to, methyl, ethyl, propyl, isopropyl, butyl, isobutyl, tert-butyl, sec-butyl, methylated butyl groups, pentyl, tert pentyl, sec-pentyl, and methylated pentyl groups. As used herein the term “unsaturated lower alkyl” means a lower alkyl group as defined supra, wherein from one to two double bonds are present in the unsaturated lower alkyl substituent. A preferred unsaturated lower alkyl is —CH₂-CH═CH₂. The term “lower alkyl-C₃-C₅ cycloalkyl” refers to C₁-C₆ alkyl substituted with a C₃-C₅ cycloalkyl group. A preferred lower alkyl-C₃-C₅ cycloalkyl group is —CH₂— -cyclopropyl; wherein the group is attached to the cryptophycin core structure at R⁹ via the CH₂.

As used herein “epoxide ring” means a three-membered ring whose backbone consists of two carbons and an oxygen atom. As used herein, “aziridine ring” means a three-membered ring whose backbone consists of two carbon atoms and a nitrogen atom. As used herein “sulfide ring” means a three-membered ring whose backbone consists of two carbon atoms and a sulfur atom. As used herein “episulfide ring” means a three-membered ring whose backbone consists of two carbon atoms and a sulfur atom. As used herein “sulfate group” means a five membered ring consisting of a carbon-carbon-oxygen-sulfur-oxygen backbone with two additional oxygen atoms connected to the sulfur atom. As used herein “cyclopropyl ring” means a three member ring whose backbone consists of three carbon atoms. As used herein, “monoalkylphosphate ring” means a five membered ring consisting of a carbon-carbon-oxygen-phosphorous-oxygen backbone with two additional oxygen atoms, one of which bears a lower alkyl group, connected to the phosphorous atom.

As used herein, “simple unsubstituted aromatic group” refers to common aromatic rings having (4n+2)p electrons in a monocyclic conjugated system, for example, but not limited to: phenyl, furyl, pyrrolyl, thienyl, pyridyl and the like, or a bicyclic conjugated system, for example but not limited to indolyl or naphthyl.

As used herein “simple substituted aromatic group” refers to a phenyl group substituted with a single group selected from the group consisting of halogen and lower alkyl group.

As used herein, “simple unsubstituted heteroaromatic group” refers to heteroaromatic rings which contain one or more non-carbon members selected from the group consisting of oxygen, nitrogen, and sulfur. This definition is included within the definition “simple unsubstituted aromatic group”.

As used herein, “simple substituted heteroaromatic group” refers to heteroaromatic rings substituted with a single group selected from the group consisting of halogen and lower alkyl group. This definition is included within the definition “simple substituted aromatic group”.

As used herein, “halogen” or “halo” refers to those members of the group on the periodic table historically known as halogens such as Cl, F, Br, I. Methods of halogenation include, but are not limited to, the addition of hydrogen halides, substitution at high temperature, photohalogenation, etc., and such methods are known to the skilled artisan.

As used herein, the term “mammal” shall refer to the Mammalia class of higher vertebrates. The term “mammal” includes, but is not limited to, a human. The term “treating” as used herein includes prophylaxis of the named condition or amelioration or elimination of the condition once it has been established. The cryptophycin compounds claimed herein can be useful for veterinary health purposes as well as for the treatment of a human patient.

Some embodiments of this invention are set forth in the following tabular form. The invention is in no way limited to the features described below:

A) R⁸ is ethyl, propyl, isopropyl, butyl, isobutyl or isopentyl;

B) R⁷ is ethyl, propyl, isopropyl, butyl, isobutyl, pentyl, or isopentyl;

C) R³ is ethyl, propyl, is isopropyl, butyl, isobutyl, pentyl, or isopentyl;

D) R⁹ is methyl, ethyl, propyl, butyl, isobutyl, pentyl, or isopentyl;

E) R¹⁰ is methyl, ethyl, propyl, butyl, isobutyl, pentyl, or isopentyl;

F) a cryptophycin compound wherein at least one of the groups selected from the group consisting of C-3, C-6, C-7, C-10, C-16, C-17, and C-18 has R stereochemistry (numbering as set forth in Formula I supra.);

G) a cryptophycin compound wherein at least one of the groups selected from the group consisting of C-3, C-6, C-7, C-10, C-16, C-17, and C-18 has S stereochemistry (numbering as set forth in Formula I supra.);

H) Ar is phenyl with a substituent selected from the group consisting of hydrogen, halogen, and lower alkyl;

I) a compound wherein the C-7 substituent is R configuration;

J) a compound wherein the C-7 substituent is S configuration;

K) Rll is lower alkyl;

L) a compound wherein R⁷, R⁸, R⁹, and R¹⁰ are each hydrogen; and R¹ and R² form an epoxide;

M) R⁷, R⁸ are each hydrogen;

N) R⁷ and R⁸ are each selected from hydrogen and CH₃;

O) R¹ and R² form an epoxide ring;

P) R⁴ and R⁵ form a double bond;

Q) R⁶ is substituted benzyl wherein one substituent is a halogen and one is an OR¹² group wherein R² is lower alkyl;

R) n is 0; R⁶ is substituted benzyl wherein one substituent is a halogen and one is an OR¹² group wherein R¹² is lower alkyl;

S) a compound of Formula I is used for disruption of a microtubulin system;

T) a compound of Formula I is used as an anti-neoplastic agent;

U) a compound of Formula I is used for the treatment of cancer in a mammal;

V) R⁶ is Formula III′ and is para hydroxy substituted;

W) R⁶ is selected from the group consisting of

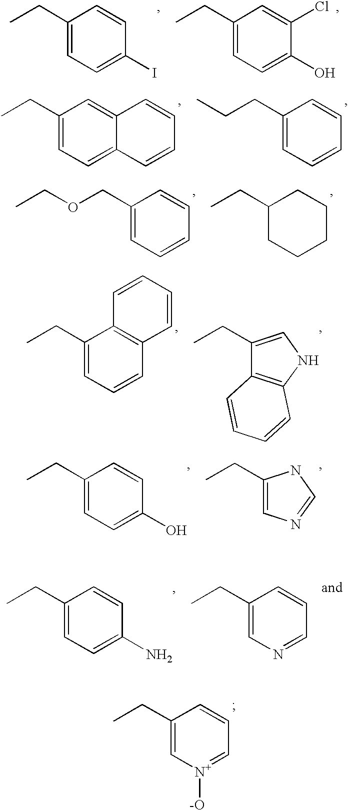

X) Z is —(CH₂)ₙ— wherein n is 0;

Y) Z is —(CH₂)ₙ— wherein n is 2;

Z) Z is —(CH₂)ₙ— wherein n is 1;

AA) R⁶ is Formula III′;

BB) R⁶ is Formula III″;

CC) R⁶ is C₃-C₆ cycloalkyl;

DD) R⁶ is selected from the group consisting of B-ring heteroaromatic, substituted heteroaromatic, B-ring alkyl, cycloalkyl, substituted cycloalkyl, Formula III′ and Formula III″;

EE) at least one of R¹⁵, R¹⁶, and R¹⁷ is selected from the group consisting of SCH₂phenyl, NH₂, CO, CONH₂, CO₂H, PO₃H₂, and SO₂R²¹; wherein R²¹ is selected from hydrogen and C₁-C₃ alkyl;

FF) Ar is phenyl;

GG) Ar is phenyl substituted with one or two from the group consisting of OH, OCH₃, halo, and methyl; and

HH) Ar is naphthyl;

II) R⁶ has a Z wherein the first carbon of the Z group is

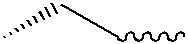

with respect to the point of attachment to the cryptophycin molecule;

JJ) R⁶ is a heteroaromatic ring;

KK) R⁷ is selected from the group consisting of N(CH₃)₂, CH₂N(CH₃)₂;

LL) R⁷ is CH₂OCH₃;

MM) R⁷ is cyclopropyl;

NN) R⁹ is CH₂cyclopropyl;

OO) R⁹ is CH₂CH═CH₂.

To further illustrate, but to no way limit, the compounds contemplated herein, the following table of especially preferred compounds is provided: A compound wherein R³ is CH₃; R⁴ and R⁵ together form a second bond; R¹⁴ is hydrogen; R³⁰ is hydrogen; R⁷ and R⁸ are each methyl; R¹⁰ is hydrogen; R⁹ is —CH₂CH(CH₃)₂; X and Y are each O; Ar is phenyl; and

| R¹ | R² | R⁶ |
|---|---|---|
| together form a double bond | | 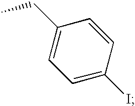 |
| together form an epoxide | | 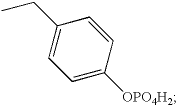 |
| together form an epoxide | | 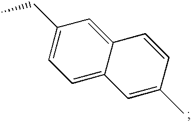 |
| together form a double bond | | 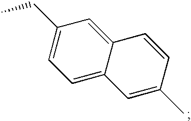 |
| Cl | OH | 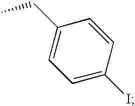 |
| Cl | OH | 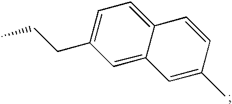 |
| together form a double bond | | 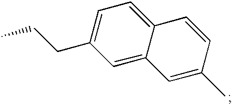 |
| together form an epoxide | | 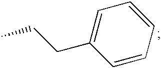 |
| Cl | OH | 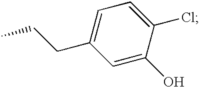 |
| together form a double bond | | 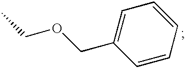 |
| Cl | OH | 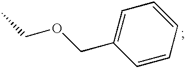 |
| together form a double bond | | 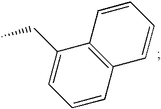 |
| together form an epoxide | | 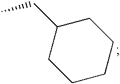 |
| Cl | OH | 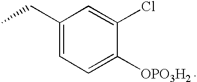 |

Additional preferred compounds are those named above except that Ar is

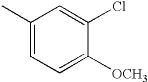

instead of phenyl. Further preferred compounds are those named above except that Ar is

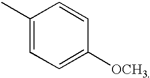

Some preferred characteristics of this invention are set forth in the following tabular form wherein the features may be independently selected to provide preferred embodiments of this invention. The invention is in no way limited to the features described below:

A) R⁸ is ethyl, propyl, isopropyl, butyl, isobutyl or isopentyl;

B) R⁷ is ethyl, propyl, isopropyl, butyl, isobutyl, pentyl, or isopentyl;

C) R³ is ethyl, propyl, is isopropyl, butyl, isobutyl, pentyl, or isopentyl;

D) R⁹ is methyl, ethyl, propyl, butyl, isobutyl, pentyl, or isopentyl;

E) R¹⁰ is methyl, ethyl, propyl, butyl, isobutyl, pentyl, or isopentyl;

F) a cryptophycin compound wherein at least one of the groups selected from the group consisting of C-3, C-6, C-7, C-10, C-16, C-17, and C-18 has R stereochemistry (numbering as set forth in Formula I supra);

G) a cryptophycin compound wherein at least one of the groups selected from the group consisting of C-3, C-6, C-7, C-10, C-16, C-17, and C-18 has S stereochemistry (numbering as set forth in Formula I supra.);

H) Ar is phenyl with a substituent selected from the group consisting of hydrogen, halogen, and lower alkyl;

I) a compound wherein the C-7 substituent is R configuration;

J) a compound wherein the C-7 substituent is S configuration;

K) R¹¹ is lower alkyl;

L) R¹ and R² form an epoxide ring;

M) R⁴ and R⁵ from a double bond;

N) n is 0; R⁶ is substituted benzyl wherein one substituent is a halogen and one is an OR¹² group wherein R¹² is lower alkyl;

0) n is 0; R⁶ is substituted benzyl wherein one substituent is a halogen and one is an OR¹² group wherein R¹² is lower alkyl;

P) a compound of Formula I is used for disruption of a microtubulin system;

Q) a compound of Formula I is used as an anti-neoplastic agent;

R) a compound of Formula I is used for the treatment of cancer in a mammal;

S) R⁶ is Formula III′ and is para hydroxy substituted;

T) R⁶ is selected from the group consisting of

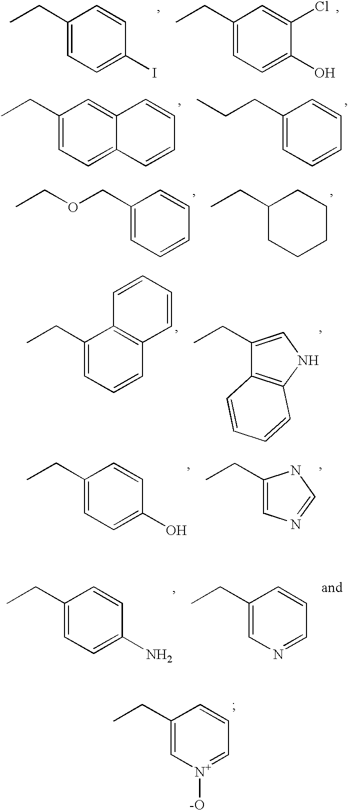

U) Z is —(CH₂)ₙ— wherein n is 0;

V) Z is —(CH₂)ₙ— wherein n is 2;

W) Z is —(CH₂)ₙ— wherein n is 1;

X) R⁶ is Formula III′;

Y) R⁶ is Formula III″;

Z) R⁶ is selected from the group consisting of B-ring heteroaromatic, substituted heteroaromatic, B-ring alkyl, cycloalkyl, substituted cycloalkyl, Formula III′ and Formula III″;

AA) Ar is phenyl;

BB) Ar is phenyl substituted with one or two from the group consisting of OH, OCH₃, halo, and methyl; and

CC) Ar is naphthyl;

DD) R⁶ has a Z wherein the first carbon of the Z group is

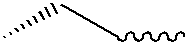

with respect to the point of attachment to the cryptophycin molecule;

EE) R⁶ is a heteroaromatic ring;

FF) R⁹ is CH₂cyclopropyl;

GG) R⁹ is CH₂CH═CH₂;

HH) R⁷ and R⁸ combine to form a spiro group;

II) R⁷ and R⁸ combine to form a cyclopropyl;

JJ) Y is 0;

KK) R⁶ is selected from the group consisting of benzyl, hydroxybenzyl, alkoxybenzyl, halohydroxybenzyl, dihalohydroxybenzyl, haloalkoxybenzyl, and dihaloalkoxybenzyl.

The present invention provides a method of alleviating a pathological condition caused by hyperproliferating mammalian cells comprising administering to a subject an effective amount of a pharmaceutical or veterinary composition disclosed herein to inhibit proliferation of the cells. In a preferred embodiment of this invention, the method further comprises administering to the subject at least one additional therapy directed to alleviating the pathological condition. In a preferred embodiment of the present invention, the pathological condition is characterized by the formation of neoplasms. In a further preferred embodiment of the present invention, the neoplasms are selected from the group consisting of mammary, small-cell lung, non-small-cell lung, colorectal, leukemia, melanoma, pancreatic adenocarcinoma, central nervous system (CNS), ovarian, prostate, sarcoma of soft tissue or bone, head and neck, gastric which includes pancreatic and esophageal, stomach, myeloma, bladder, renal, neuroendocrine which includes thyroid and non-Hodgkin's disease and Hodgkin's disease neoplasms.

As used herein “neoplastic” refers to a neoplasm, which is an abnormal growth, such growth occurring because of a proliferation of cells not subject to the usual limitations of growth. As used herein, “anti-neoplastic agent” is any compound, composition, admixture, co-mixture, or blend which inhibits, eliminates, retards, or reverses the neoplastic phenotype of a cell.

As used herein “hyperproliferation” or is the overproduction of cells in response to a particular growth factor. “Hyperproliferative disorders” are disease in which the cells overproduce in response to a particular growth factor. Examples of such “hyperproliferative disorders” include diabetic retinopathy, psoriasis, endometriosis, cancer, macular degenerative disorders and benign growth disorders such as prostate enlargement.

Anti-mitotic agents may be classified into three groups on the basis of their molecular mechanism of action. The first group consists of agents, including colchicine and colcemid, which inhibit the formation of microtubules by sequestering tubulin. The second group consists of agents, including vinblastine and vincristine, which induce the formation of paracrystalline aggregates of tubulin. Vinblastine and vincristine are well known anticancer drugs: their action of disrupting mitotic spindle microtubules preferentially inhibits hyperproliferative cells. The third group consists of agents, including taxol, which promote the polymerization of tubulin and thus stabilizes microtubules.

The exhibition of drug resistance and multiple-drug resistance phenotype by many tumor cells and the clinically proven mode of action of anti-microtubule agents against neoplastic cells necessitates the development of anti-microtubule agents cytotoxic to non-drug resistant neoplastic cells as well as cytotoxic to neoplastic cells with a drug resistant phenotype.

Chemotherapy, surgery, radiation therpy, therapy with biological response modifiers, and immunotherapy are currently used in the treatment of cancer. Each mode of therapy has specific indications which are known to those of ordinary skill in the art, and one or all may be employed in an attempt to achieve total destruction of neoplastic cells. Moreover, combination chemotherapy, chemotherapy utilizing compounds of Formula I in combination with other neoplastic agents, is also provided by the subject invention as combination therapy is generally more effective than the use of a single anti-neoplastic agent. Thus, a further aspect of the present invention provides compositions containing a therapeutically effective amount of at least one compound of Formula I, including the non-toxic addition salts thereof, which serve to provide the above recited benefits. Such compositions can also be provided together with physiologically tolerable liquid, gel, or solid carriers, diluents, adjuvants and excipients. Such carriers, adjuvants, and excipients may be found in the U.S. Pharmacopeia, Vol. XXII and National Formulary vol XVII, U.S. Pharmacopeia Convention, Inc. Rockville, Md. (1989). Additional modes of treatment are provided in AHFS Drug Information, 1993 e. by the American Hospital Formulary Service, pp. 522-660. Each of these references are well known and readily available to the skilled artisan.

The present invention further provides a pharmaceutical composition used to treat neoplastic disease containing at least one compound of Formula I and at least one additional anti-neoplastic agent. Anti-neoplastic agents which may be utilized in combination with Formula I compounds include those provided in the Merck Index 11, pp 16-17, Merck & Co., Inc. (1989). The Merck Index is widely recognized and readily available to the skilled artisan.

In a further embodiment of this invention, antineoplastic agents may be antimetabolites which may include but are in no way limited to those selected from the group consisting of methotrexate, 5-fluorouracil, 6-mercaptopurine, cytosine, arabinoside, hydroxyurea, and 2-chlorodeoxyadenosine. In another embodiment of the present invention, the anti-neoplastic agents contemplated are alkylating agents which may include but are in no way limited to those selected from the group consisting of cyclophosphamide, mephalan, busulfan, paraplatin, chlorambucil, and nitrogen mustard. In a further embodiment, the anti-neoplastic agents are plant alkaloids which may include but are in no way limited to those selected from the group consisting of vincristine, vinblastine, taxol, and etoposide. In a further embodiment, the anti-neoplastic agents contemplated are antibiotics which may include, but are in no way limited to those selected from the group consisting of doxorubicin, daunorubicin, mitomycin C, and bleomycin. In a further embodiment, the anti-neoplastic agents contemplated are hormones which may include, but are in no way limited to those selected from the group consisting of calusterone, diomostavolone, propionate, epitiostanol, mepitiostane, testolactone, tamoxifen, polyestradiol phosphate, megesterol acetate, flutamide, nilutamide, and trilotane.

In a further embodiment, the anti-neoplastic agents contemplated include enzymes which may include, but are in no way limited to those selected from the group consisting of L-Asparginase and aminoacridine derivatives such as, but not limited to, amsacrine. Additional anti-neoplastic agents include those provided by Skeel, Roland T., “Antineoplastic Drugs and Biologic Response Modifier: Classification, Use and Toxicity of Clinically Useful Agents” Handbook of Cancer Chemotherapy (3rd ed.), Little Brown & Co. (1991).

These compounds and compositions can be administered to mammals for veterinary use. For example, domestic animals can be treated in much the same way as a human clinical patient. In general, the dosage required for therapeutic effect will vary according to the type of use, mode of administration, as well as the particularized requirements of the individual hosts. Typically, dosages will range from about 0.001 to 1000 mg/kg, and more usually 0.01 to 10 mg/kg of the host body weight. Alternatively, dosages within these ranges can be administered by constant infusion over an extended period of time, usually exceeding 24 hours, until the desired therapeutic benefits are obtained. Indeed, drug dosage, as well as route of administration, must be selected on the basis of relative effectiveness, relative toxicity, growth characteristics of tumor and effect of Formula I compound on cell cycle, drug pharmacokinetics, age, sex, physical condition of the patient and prior treatment, which can be determined by the skilled artisan.

The compound of Formula I, with or without additional anti-neoplastic agents, may be formulated into therapeutic compositions as natural or salt forms. Pharmaceutically acceptable non-toxic salts include base addition salts which may be derived from inorganic bases such as for example, sodium, potassium, ammonium, calcium, or ferric hydroxides, and such organic bases as isopropylamine, trimethylamine, 2-ethylamino ethanol, histidine, procaine, and the like. Such salts may also be formed as acid addition salts with any free cationic groups and will generally be formed with inorganic acids such as for example, hydrochloric or phosphoric acids or organic acids such as acetic, oxalic, tartaric, mandelic, and the like. Additional excipients which further the invention are provided to the skilled artisan for example in the U.S. Pharmacopeia.

The suitability of particular carriers for inclusion in a given therapeutic composition depends on the preferred route of administration. For example, anti-neoplastic compositions may be formulated for oral administration. Such compositions are typically prepared as liquid solution or suspensions or in solid forms. Oral formulation usually include such additives as binders, fillers, carriers, preservatives, stabilizing agents, emulsifiers, buffers, mannitol, lactose, starch, magnesium stearate, sodium saccharin, cellulose, magnesium carbonate, and the like. These compositions may take the form of solutions, suspensions, tablets, pills, capsules, sustained release formulations, or powders, and typically contain 1% to 95% of active ingredient. More preferably, the composition contains from about 2% to about 70% active ingredient.

Compositions of the present invention may be prepared as injectables, either as liquid solutions, suspensions, or emulsions; solid forms suitable for solution in or suspension in liquid prior to injection. Such injectables may be administered subcutaneously, intravenously, intraperitoneally, intramuscularly, intrathecally, or intrapleurally. The active ingredient or ingredients are often mixed with diluents, carriers, or excipients which are physiologically tolerable and compatible with the active ingredient(s). Suitable diluents and excipients are for example, water, saline, dextrose, glycerol, or the like and combinations thereof. In addition, if desired, the compositions may contain minor amounts of auxilary substances such as wetting or emulsifying agents, stabilizing or pH buffering agents.

The invention further provides methods for using Formula I compounds to inhibit the proliferation of mammalian cells by contacting these cells with a Formula I compound in an amount sufficient to inhibit the proliferation of the mammalian cell. A preferred embodiment is a method to inhibit the proliferation of hyperproliferative mammalian cells. For purposes of this invention “hyperproliferative mammalian cells” are mammalian cells which are not subject to the characteristic limitations of growth (programmed cell death for example). A further preferred embodiment is when the mammalian cell is human. The invention further provides contacting the mammalian cell with at least one Formula I compound and at least one anti-neoplastic agent. The types of anti-neoplastic agents contemplated are discussed supra.

The invention further provides methods for using a compound of Formula I to inhibit the proliferation of hyperproliferative cells with drug-resistant phenotypes, including those with multiple drug-resistant phenotypes, by contacting said cell with a compound of Formula I in an amount sufficient to inhibit the proliferation of a hyperproliferative mammalian cell. A preferred embodiment is when the mammalian cell is human. The invention further provides contacting a Formula I compound and at least one additional anti-neoplastic agent, discussed supra.

The invention provides a method for alleviating pathological conditions caused by hyperproliferating mammalian cells for example, neoplasia, by administering to a subject an effective amount of a pharmaceutical composition containing Formula I compound to inhibit the proliferation of the hyperproliferating cells. As used herein “pathological condition” refers to any pathology arising from the proliferation of mammalian cells that are not subject to the normal limitations of growth. Such proliferation of cells may be due to neoplasms as discussed supra.

In a further preferred embodiment the neoplastic cells are human. The present invention provides methods of alleviating such pathological conditions utilizing a compound of Formula I in combination with other therapies, as well as other anti-neoplastic agents.

The effectiveness of the claimed compounds can be assessed using standard methods known to the skilled artisan. Examples of such methods are as follows:

The compounds are screened for minimum inhibitory concentrations against KB, a human nasopharyngeal carcinoma cell line, LoVo, a human colorectal adenocarcinoma cell line using The Corbett assay, see Corbett, T. H. et al. Cytotoxic Anticancer Drugs: Models and Concepts for Drug Discovery and Development, pp 35-87, Kluwer Academic Publishers: Norwell, 1992. see also, Valeriote, et al. Discovery and Development of Anticancer Agents; Kluwer Academic Publishers, Norwell, 1993 is used for the evaluation of compounds.

The most active compounds are further evaluated for cytotoxicity against four different cell types, for example a murine leukemia, a murine solid tumor, a human solid tumor, and a low malignancy fibroblast using the Corbett assay.

The compounds are further evaluated against a broad spectrum of murine and human tumors implanted in mice, including drug resistant tumors.

Tumor burden (T/C) (mean tumor burden in treated animals versus mean tumor burden in untreated animals) are used as a further assessment. T/C values that are less than 42% are considered to be active by National Cancer Institute Standards; T/C values less than 10% are considered to have excellent activity and potential clinical activity by National Cancer Institute standards.

Materials

Vinblastine, cytochalasin B, tetramethylrhodamine isothiocyanate (TRITC)-phalloidin, sulforhodamine B (SRB) and antibodies against β-tubulin and vimentin are commercially available from recognized commercial vendors. Basal Medium Eagle containing Earle's salts (BME) and Fetal Bovine Serum (FBS) are also commercially available.

Cell Lines

The Jurkat T cell leukemia line and A-10 rat aortic smooth muscle cells are obtained from the American Type Culture Collection and are cultured in BME containing 10% FBS and 50 μg/mL gentamycin sulfate. Human ovarian carcinoma cells (SKOV3) and a sub-line which has been selected fro resistance to vinblastine (SKVLB1) were a generous gift from Dr. Victor Ling of the Ontario Cancer Institute. Both cell lines are maintained in BME containing 10% FBS and 50 μg/mL gentamycin sulfate. Vinblastine is added to a final concentration of 1 μg/mL to SKVLB1 cells 24 hours after passage to maintain selection pressure for P-glycoprotein-overexpressing cells.

Cell Proliferation and Cycle Arrest Assays

Cell proliferation assays are performed as described by Skehan et al. For Jurkat cells, cultures are treated with the indicated drugs as described in Skehan and total cell numbers are determined by counting the cells in a hemacytometer. The percentage of cells in mitosis are determined by staining with 0.4% Giemsa in PBS followed by rapid washes with PBS. At least 1000 cells per treatment are scored for the presence of mitotic figures and the mitotic index is calculated as the ration of the cells with mitotic figures to the total number of cells counted.

Immunofluorescence Assays

A-10 cells are grown to near-confluency on glass coverslips in BME/10% FBS. Compounds in PBS are added to the indicated final concentrations and cells are incubated for an additional 24 hours. For the staining of microtubules and intermediate filaments, the cells are fixed with cold methanol and incubated with PBS containing 10% calf serum to block nonspecific binding sites. Cells are then incubated at 37° C for 60 min. with either monoclonal anti-S-tubulin or with monoclonal anti-vimentin at dilutions recommended by the manufacturer. Bound primary antibodies are subsequently visualized by a 45-minute incubation with fluorescein-conjugated rabbit antimouse IgG. The coverslips are mounted on microscope slides and the fluorescence patterns are examined and photographed using a Zeiss Photomicroscope Ill equipped with epifluorescence optics for fluorescein. For staining of microfilaments, cells are fixed with 3% paraformaldehyde, permeabilized with 0.2% Triton X-100 and chemically reduced with sodium borohydride (1 mg/ML). PBS containing 100 nM TRITC-phalloidin is then added and the mixture is allowed to incubate for 45 min. at 37° C. The cells are washed rapidly with PBS before the coverslips are mounted and immediately photographed as described above.

Effects of Cryptophycins and Vinblastine on Jurkat Cell Proliferation and Cell Cycle

Dose-response curves for the effects of cryptophycin compounds and vinblastine on cell proliferation and the percentage of cells in mitosis are determined.

Effects of Cytochalasin B, Vinblastine and Cryptophycins on the Cytoskeleton

Aortic smooth muscle (A-10) cells are grown on glass coverslips and treated with PBS, 2 μM cytochalasin B, 100 nM vinblastine or 10 nM cryptophycin compounds. After 24 hours, microtubules and vimentin intermediate filaments are visualized by indirect immunofluorescence and microfilaments are stained using TRITC-phalloidin. The morphological effects of each drug is examined. Untreated cells displayed extensive microtubule networks complete with perinuclear microtubule organizing centers. Vimentin intermediate filaments were also evenly distributed throughout the cytoplasm, while bundles of microfilaments were concentrated along the major axis of the cell. Cytochalasin B caused complete depolymerization of microfilaments along with the accumulation of paracrystalline remnants. This compound did not affect the distribution of either microtubules or intermediate filaments. The cryptophycin treated microtubules and vimentin intermediates are observed for depletion of microtubules, and collapse of rimentin intermediate filaments.

Effects of Cryptophycins and Vinblastine on Taxol-stabilized Microtubules

A-10 cells are treated for 3 hours with 0 or 10 μM taxol before the addition of PBS, 100 nM vinblastine or 10 nM cryptophycin compound. After 24 hours, microtubule organization is examined by immunofluorescence as described above. Compared with those in control cells, microtubules in taxol-treated cells were extensively bundled, especially in the cell polar regions. As before, vinblastine caused complete depolymerization of microtubules non-pretreated cells. However, pretreatment with taxol prevented microtubule depolymerization in response to vinblastine. Similarly, microtubules pretreated with taxol are observed with cryptophycin treatment.

Reversibility of Microtubule Depolymerization by Vinblastine and Cryptophycin

A-10 cells are treated with either 100 nM vinblastine or 10 nM cryptophycins for 24 hr., resulting in complete microtubule depolymerization. The cells are then washed and incubated in drug-free medium for periods of 1 hour or 24 hours. Microtubules repolymerized rapidly after the removal of vinblastine, showing significant levels of microtubules after 1 hour and complete morphological recovery by 24 hour. Cells are visualized for microtubule state after treatment with a cryptophycin compound of this invention at either 1 hour or 24 hours after removal of the cryptophycin compounds.

Effects of Combinations of Vinblastine and Cryptophycins on Cell Proliferation

SKOV3 cells are treated with combinations of cryptophycins and vinblastine for 48 hours. The percentages of surviving cells are then determined and the IC₅₀s for each combination is calculated.

Toxicity of Cryptophycins, Vinblastine and Taxol toward SKOV3 and SKVLB1 Cells

SKVLB1 cells are resistant to natural product anticancer drugs because of their over expression of P-glycoprotein. The abilities of taxol, vinblastine and cryptophycin compounds to inhibit the growth of SKOV3 and SKVLB1 cells are observed. Taxol caused dose-dependent inhibition of the proliferation of both cell lines with IC₅₀s for SKOV3 and SKVLB1 cells of 1 and 8000 nM, respectively. Vinblastine also inhibited the growth of both cell lines, with IC₅₀s of 0.35 and 4200 nM for SKOV3 and SKVLB1 cells, respectively. Cryptophycins compounds of this invention demonstrate activity with an IC₅₀S of from about 1 to about 1000 pm for SKOV3 and SKVLB1 cells.

Thus it can be demonstrated that the present invention provides novel cryptophycin compounds which are potent inhibitors of cell proliferation, acting by disruption of the microtubule network and inhibition of mitosis. These studies can illustrate that cryptophycin compounds disrupt microtubule organization and thus normal cellular functions, including those of mitosis.

Classic anti-microtubule agents, such as colchicine and Vinca alkaloids, arrest cell division at mitosis. It seems appropriate to compare the effect of one of these agents on cell proliferation with the cryptophycin compounds. For this purpose, the Vinca alkaloid vinblastine was selected as representative of the classic anti-microtubule agents. Accordingly, the effect of cryptophycin compounds and vinblastine on the proliferation and cell cycle progression of the Jurkat T-cell leukemia cell line is compared.

Since antimitotic effects are commonly mediated by disruption of microtubules in the mitotic spindles, the effects of cryptophycin compounds on cytoskeletal structures are characterized by fluorescence microscopy. Immunofluorescence staining of cells treated with either a cryptophycin compound or vinblastine demonstrate that both compounds cause the complete loss of microtubules. Similar studies with SKOV3 cells can show that the anti-microtubule effects of cryptophycin compounds are not unique to the smooth muscle cell line.

GC3 human Colon Carcinoma Screen

Selected wells of a 96 well plate were seeded with GC3 human colon carcinoma cells (1×10 cells in 100 μl assay medium/well) twenty four hours prior to test compound addition. Cell free assay medium was added to other select wells of the 96 well plate. The assay medium (RPMI-1640 was the medium used; however, any medium that will allow the cells to survive would be acceptable) was supplemented with 10% dialyzed fetal bovine serum and 25 mM HEPES buffer.

The test compound was stored in an amber bottle prior to testing. Fresh dimethylsulfoxide stock solution (200 μg/ml) was prepared immediately prior to preparation of test sample dilutions in phosphate-buffered saline (PBS). A dilution of 1:20 dimethylsulfoxide solution in PBS was prepared such that the final concentration was 10 μg/ml. Serial 1:3 dilutions using PBS (0.5ml previous sample of 1 ml PBS) were prepared. Falcon 2054 tubes were used for the assay.

A 10 ul sample of each dilution of test compound was added in triplicate to wells of GC3 plates. The plates were incubated for 72 hours at about 37 C. A 10 μl sample of stock 3-[4,5-dimethyl-2-yl]-2,5-diphenyltetrazolium bromide salt (“MTT” 5 mg/ml in PBS) was added to each well. The plates were incubated for about an hour at 37 C. The plates were centrifuged, media was decanted from the wells and 100 μl acid-isopropanol (0.04 N HCl in isopropanol) was added to each well. The plate was read within one hour using a test wavelength of 570 nm (SpectraMax reader).

Evaluation of compounds of Formula I suggest that the compounds can be useful in the treatment methods claimed herein. Further, the compounds will be useful for disrupting the microtubule system.

Compounds of Formula I can be prepared using a compound of the Formula II

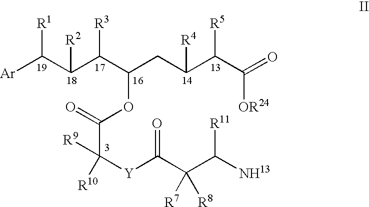

wherein

Ar, R¹, R², R³, R⁴, R⁵, R⁷, R⁸, R⁹, R¹⁰, R¹¹, and Y have the meanings set for supra in Formula I.

R¹³ is selected from the group consisting of t-butylcarbamate (BOC);

R²⁴ is selected from the group consisting of

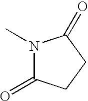

(N-hydroxysuccinimide, herein “NHS”), N-hydroxysulfosuccinimide and salts thereof, 2-nitrophenyl, 4-nitrophenyl, and 2,4-dichlorophenyl;

Compounds of Formula III

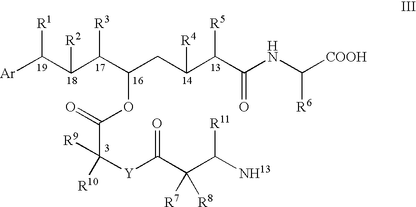

wherein the R groups and various substituents are as defined hereinbefore and throughout the specification; can be prepared by contacting a compound of the Formula IV

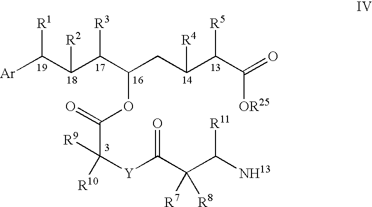

R²⁵ is an active ester substituent;

with an acid of the Formula

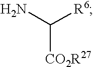

R²⁷ is selected from the group consisting of H, C₁-C₁₂ alkyl, and aryl; and a silylating agent. Bis N,O-trimethylsilyl acetamide (BSA) is an especially preferred silylating agent.

As used with regard to R²⁵ the phrase “active ester substituent” refers to a substituent which makes the OR²⁴ substituent a good leaving group. Appropriate substituents can be selected with guidance from standard reference guides, for example, “Protective Groups in Organic Chemistry”, Plenum Press, (London and New York, 1973); Greene, T. W. “Protecting Groups in Organic Synthesis”, Wiley (New York, 1981). An especially preferred R²⁵ group is N-hydroxy-succinimide. (NHS)

The processes described herein are most preferably completed in the presence of a solvent. The artisan can select an appropriate solvent for the above described process. Inert organic solvents are particularly preferred; however, under certain conditions an aqueous solvent can be appropriate. For example, if R²⁷ is hydrogen and and R¹³ is BOC an aqueous base as solvent will be effective.

When the desired R⁶ substituent in the compound of Formula I contains an amine, then the amine substituent of the R⁶ group must be protected using an amino protecting group. The artisan can readily select an appropriate amino protecting group using guidance from standard works, including, for example, “Protective Groups in Organic Chemistry”, Plenum Press, (London and New York, 1973); Greene, T. W. “Protecting Groups in Organic Synthesis”, Wiley (New York, 1981).

R²⁷ should be a group that allows for the removal of the —CO₂R²⁷ substituent using acidic, neutral, or mild basic conditions. Preferred R²⁷ groups include, but are in no way limited to, hydrogen, C₁-C₆ alkyl, trichloromethyl, trichloroethyl, and methylthiomethyl. It is especially preferred that R²⁷ is hydrogen.

To provide further guidance for the artisan, the following schemes are provided:

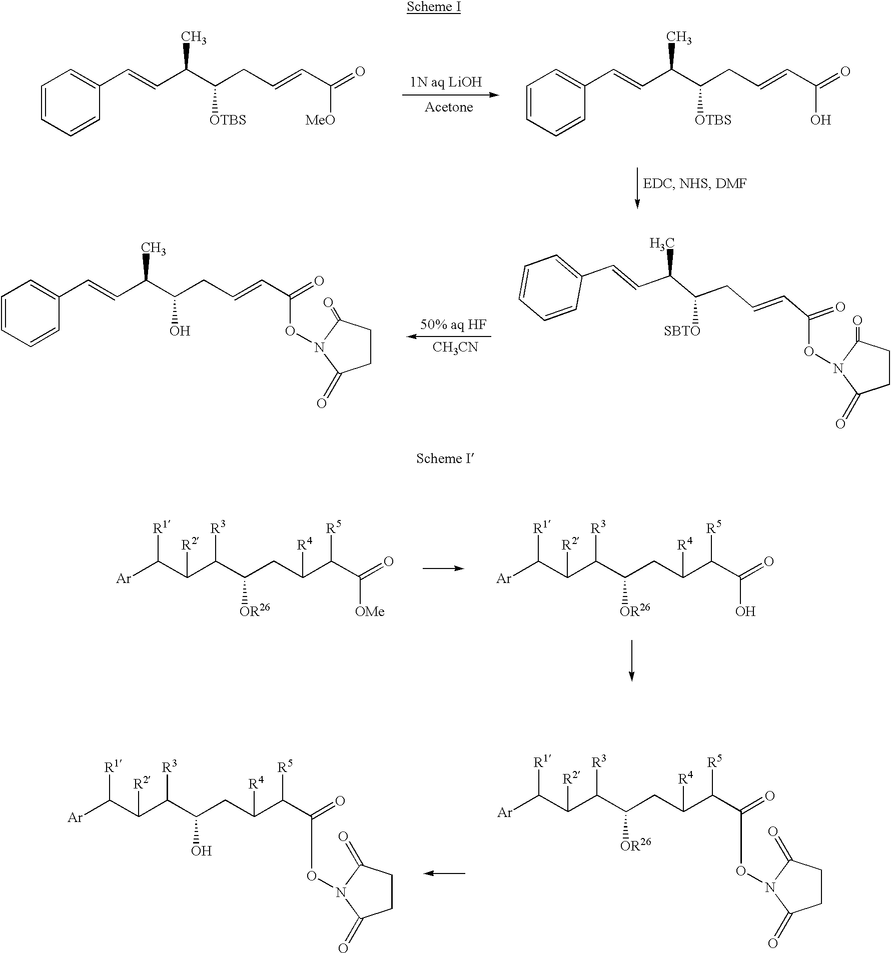

As used in Scheme I′ and throughout the specification, R¹′ is halogen, SH, amino, monoalkylamino, dialkylamino, trialkylammonium, alkylthio, dialkylsulfonium, sulfate, phosphate or a protected OH or protected SH group; R² is OH or SH; R²⁶ is an alcohol protecting group introduced during a portion of the synthetic process to protect an alcohol group which might otherwise react in the course of chemical manipulations, and is then removed at a later stage of the synthesis. Numerous reactions for the formation and removal of such a protecting groups are described in a number of standard works, including, for example, “Protective Groups in Organic Chemistry”, Plenum Press, (London and New York, 1973); Greene, T. W. “Protecting Groups in Organic Synthesis”, Wiley (New York, 1981). The skilled artisan can select an appropriate alcohol protecting group particularly with guidance provided from such works. One particularly useful alcohol protecting group is tert-butyldimethylsilyl (TBS).

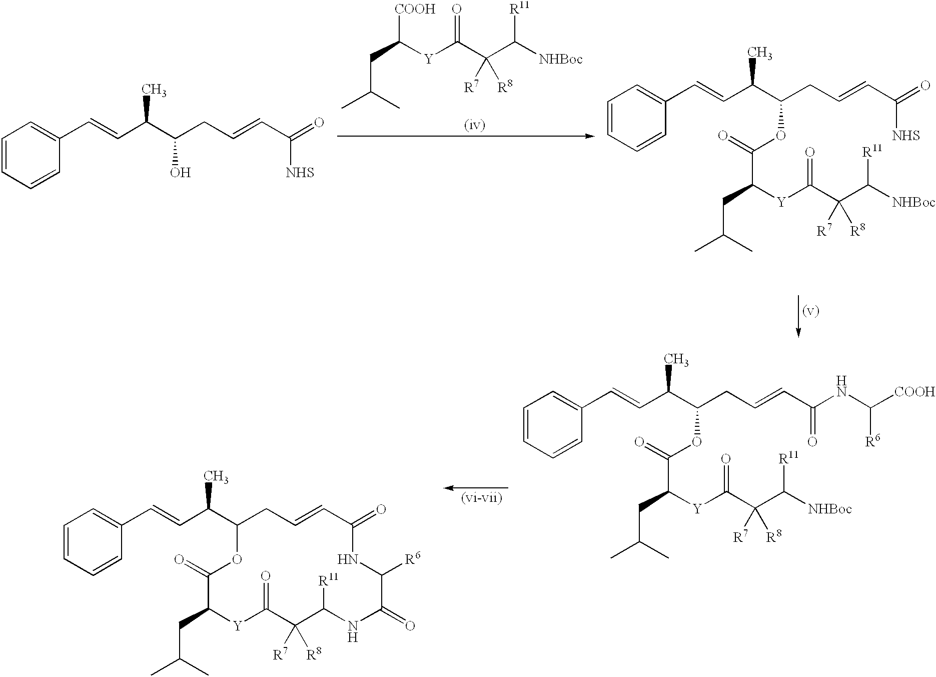

R⁶, R⁷, R⁸, R¹¹, R³⁰ and Y have the meanings defined supra.

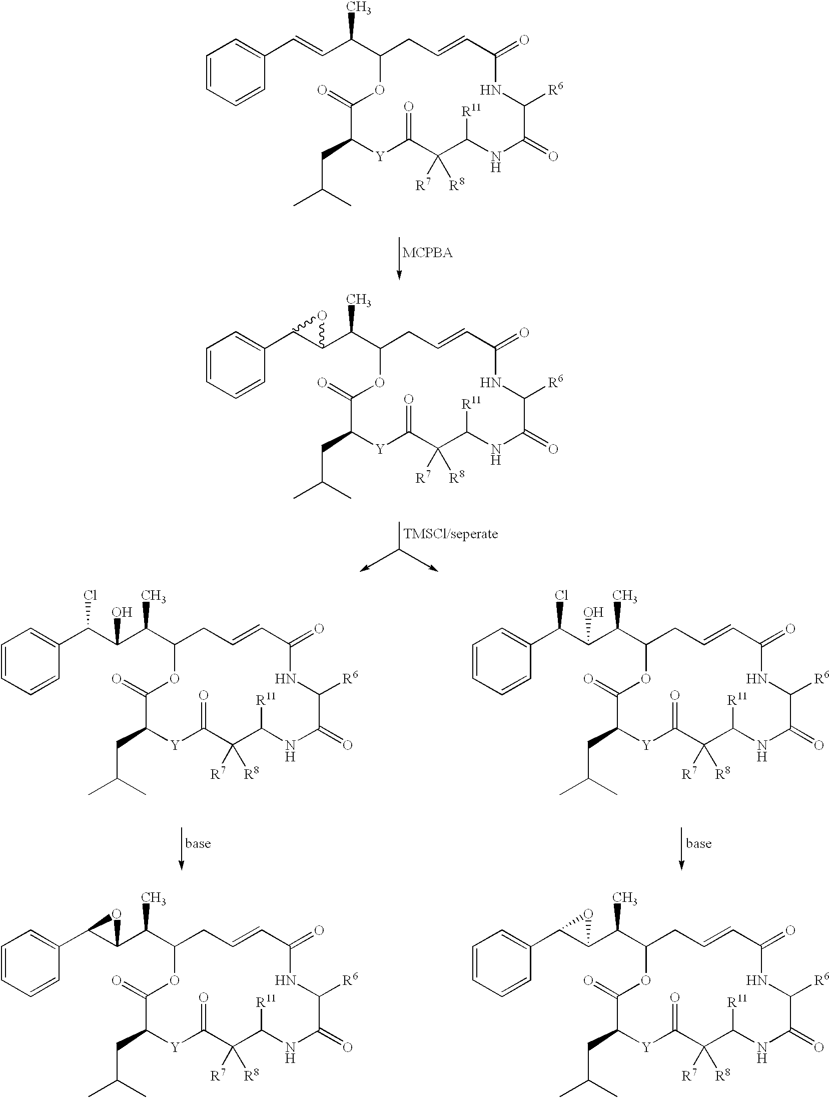

The product of the schemes provided herein can be further derivatized using standard methods to provide further cryptophycin compounds.

The artisan can utilize appropriate starting materials and reagents to prepare desired compounds using the guidance of the previous schemes and following examples. The ester starting material can be prepared, for example, as follows:

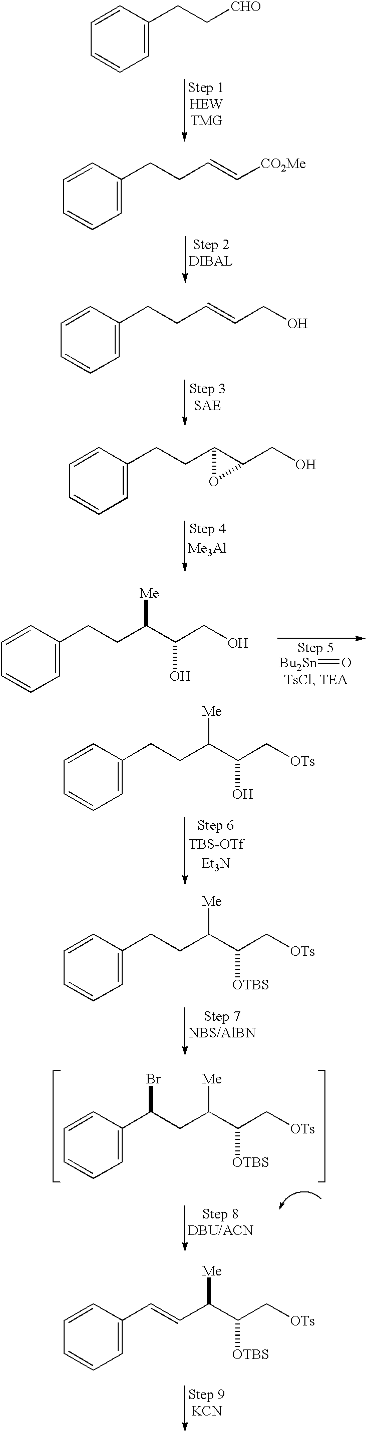

The scheme for preparing the ester is further explained by the Preparation Section herein which provides one specific application of the scheme for the convenience of the skilled artisan.

The Scheme for preparing the ester is applicable to the Ar substituents claimed herein. The scheme illustration is not intended to limit the synthesis scheme only to the phenyl ring illustrated. Rather, the artisan can broadly apply this process to provide desired starting materials for the compounds claimed herein.

The necessary reaction time is related to the starting materials and operating temperature. The optimum reaction time for a given process is, as always, a compromise which is determined by considering the competing goals of throughput, which is favored by short reaction times, and maximum yield, which is favored by long reaction times.

The effectiveness of the claimed compounds can be assessed using methods known to the skilled artisan. One such study provided the following results:

| | IC50 (nM) | |
|---|---|---|
| | GC3/C1 | HT-29 |
| 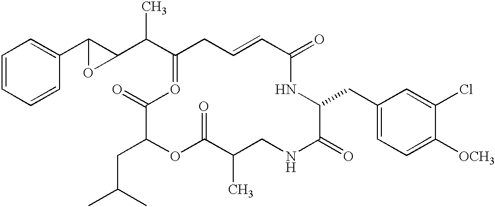 | 0.24 | |
| 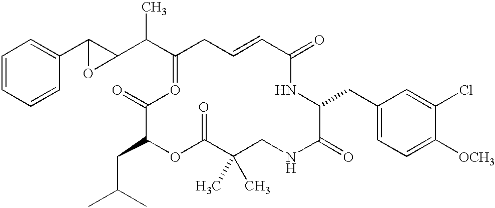 | 0.14 | |
| 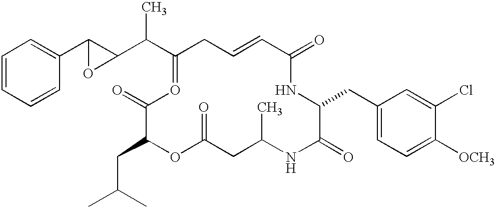 | 1.01 (1.1 nM/HT29) (16 nM/MX) | |
| 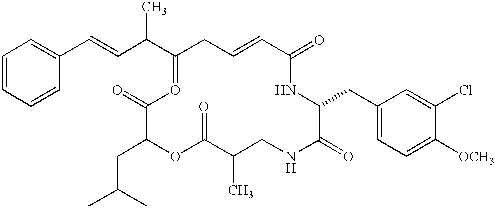 | | |
| 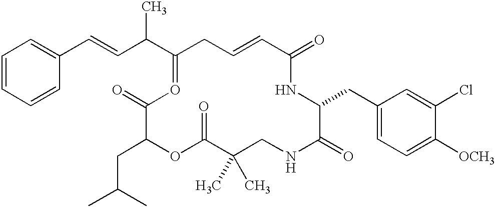 | 85 | 80 |
| 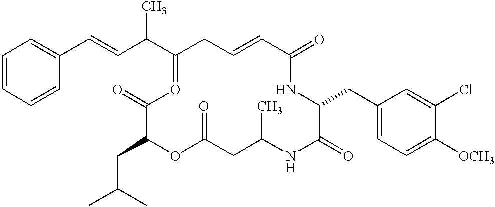 | 157 | 108 |

The compounds are screened for minimum inhibitory concentrations against KB, a human nasopharyngeal carcinoma cell line, LoVo, a human colorectal adenocarcinoma cell line. The Corbett assay, see Corbett, T. H. et al. Cytotoxic Anticancer Drugs: Models and Concepts for Drug Discovery and Development, pp 35-87, Kluwer Academic Publishers: Norwell, 1992. see also, Valeriote, et al. Discovery and Development of Anticancer Agents; Kluwer Academic Publishers, Norwell, 1993.

The most active compounds are further evaluated for cytotoxicity against four different cell types, for example a murine leukemia, a murine solid tumor, a human solid tumor, and a low malignancy fibroblast using the Corbett assay.

The compounds are further evaluated against a broad spectrum of murine and human tumors implanted in mice, including drug resistant tumors.

Tumor burden (T/C) (mean tumor burden in treated animals verses mena tumor burden in untreated animals) are used as a further assessment. T/C values that are less than 42% are considered to be active by National Cancer Institute Standards; T/C values less than 10% are considered to have excellent activity and potential clinical activity by National Cancer Institute standards.

Evaluation of compounds of Formula I suggest that the compounds can be useful in the treatment methods claimed herein. Further, the compounds will be useful for disrupting the microtubule system.

The compounds of this invention where R¹¹ is other than hydrogen can be prepared as illustrated using the following schemes.

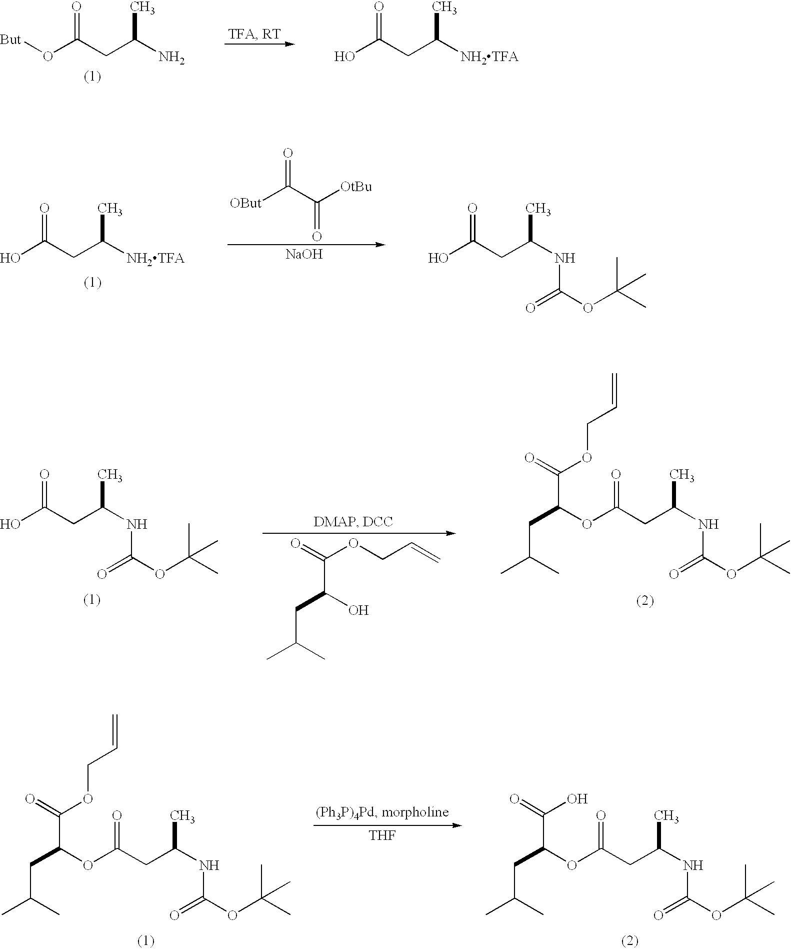

To further illustrate the invention the following examples are provided. The scope of the invention is in no way to be construed as limited to or by the following examples.

### PREPARATIVE EXAMPLE 1

Step 1. Methyl 5-Phenylpent-2(E)-enoate.

A solution of trimethyl phosphonoacetate (376 g, 417 mL, 2.07 mol) in THF (750 mL) was stirred at 0° C. in a 3 L 3-neck round bottom flask equipped with a mechanical stirrer and N₂ inlet. To the chilled solution, neat tetramethyl guanidine (239 g, 260 mL, 2.07 mol) was added dropwise via an addition funnel. The chilled clear pale yellow solution was stirred for 25 minutes at 0° C. A solution of hydrocinnamaldehyde (90%, 253 g, 248 mL, 1.9 mol) in THF (125 mL) was added dropwise to the reaction solution slowly. Upon completion of addition, the reaction was stirred for 10 h rising to room temperature. GC indicated a 95:5 ratio of product to starting material. 500 ml of water was added to the reaction vessel and the reaction stirred overnight separating into two layers. The organic layer was isolated and the aqueous layer was extracted with t-BuOMe. The organic layers were combined and dried over MgSO₄, then concentrated in vacuo to yield an orange oil. The crude product was distilled at 129° C./0.3 mm Hg yielding 360.5 g, 91.7% yield, of a clear slightly yellow oil.

EIMS m/z 190 (13; M+), 159 (410, 158 (39), 131 (90), 130 (62), 117 (22), 104 (12), 95 (57), 91 (100), 77 (21), 65 (59); HREIMS m/z 190.0998 (C₁₂H₄O₂ D −0.4 mnu); UV lmax (e) 210 (8400), 260 (230) nm; IR nmax 3027, 2949, 1723, 1658, 1454, 1319, 1203, 978, 700 cm⁻¹; ¹H NMR d (CDCl₃) 7.15-7.3 (Ph-H5;bm), 7.00 (3-H;dt, 15.6/6.6), 5.84 (2-H;dt, 15.6/1.2), 3.70 (OMe;s), 2.76 (5-H2;t, 7.2), 2.51 (4-H2; bdt, 6.6/7.2); ¹³C NMR d (CDCl3) 166.9 (1), 148.3 (3), 140.6 (Ph-1′), 128.4/128.2 (Ph2′/3′/5′6′), 126.1 (Ph 4′), 121.4 (2). 51.3 (OMe), 34.2/33.8 (4/5).

Step 2. 5-phenyl-pent-2-en-1-ol.

To a 12 L 4-neck round bottom flask equipped with a thermocouple, mechanical stirrer and N₂ inlet, a solution of enoate ester (310.5 g, 1.5 mol) in THF (1.5 L) was charged and chilled to −71° C. via a i-PrOH/CO₂ bath. To the reaction vessel, was added dropwise DIBAL (2.5 L, 1.5 M in toluene, 3.75 mol) at a rate to maintain the reaction temperature <−50° C. Upon complete addition, the reaction was stirred overnight with the reaction temperature <−50° C. TLC (3:1 Hexanes:EtOAc, SiO₂) indicated absence of starting material after 16 h. The reaction temperature was allowed to raise to −15° C. The reaction was quenched slowly with 1N HCl (150 mL). At this point the reaction setup into a gelatinous solid. A spatula was employed to breakup the the semi-solid and 1N HCl (200 mL) was added making the mixture more fluid. Concentrated HCl (625 mL) was charged to form a two phase system. The layers were separated and the product extracted with t-BuOMe. The organic layer was dried over MgSO₄ and concentrated in vacuo to yield a clear pale yellow oil, 247.8g. The crude product was distilled at 145° C./0.25mm Hg yielding 209.7g, 86.2%.

EIMS m/z 162 (1:M+) 144 (16), 129 (7), 117 (9) 108 (6), 92 (17), 91 (100), 75 (5), 65 (12), HREIMS m/z 162, 1049 (C₁₁H₁₄O, D −0.4 mmu); UV 1max (e) 206 (9900), 260 (360); IR nmax 3356, 2924, 1603, 1496, 1454, 970, 746, 700 cm⁻¹; ¹H NMR d 7.15-7.3 (Ph-H5;m), 5.70 (3-H;dt, 15.6/6.0), 5.61 (2-H;dt, 15.6/4.8), 4.02 (1-H2;d 4.8), 2.68 (5-H2; t, 7.2), 2.40 (OH;bs), 2.36 (4-H2; dt, 6.0/7.2); ¹³C NMR dl41.6 (Ph 1′), 131.8 (3), 129.5 (2), 128.3/128.2 (Ph 2′/3′/5′/6′), 125.7 (Ph 4′), 63.3 (1), 35.4/33.8 (4/5).

Step 3. (2S, 3S)-2,3-Epoxy-5-phenyl-1-pentanol.

To a 1 L 3 neck round bottom flask equipped with a mechanical stirrer, thermocouple and nitrogen inlet was added CH₂Cl₂ (350 mL), dried 4 Å molecular sieves (30 g) and L-(+)-diethyl tartrate (7.62 g, 0.037 mol). The resulting mixture was cooled to −20° C. and treated with Ti(O-i-Pr)4 (9.2 mL, 0.031 mol), followed by the addition of t-butylhydroperoxide (4.0 M in CH₂Cl₂, 182 mL, 0.78 mol) at a rate to maintain the temperature=−20° C. Upon complete addition, the reaction mixture was stirred for another 30 min, and then treated with a solution of the allylic alcohol (50 g, 0.31 mol) in CH₂Cl₂ (30 mL) at a rate to maintain the temperature=−20° C. The reaction was stirred at the same temperature for 5 h, then filtered into a solution of ferrous sulfate heptahydrate (132 g) and tartaric acid (40 g) in water (400 mL) at 0° C. The mixture was stirred for 20 min, then transferred to a separatory funnel and extracted with t-BuOMe (2×200 mL). The combined organic phase was stirred with 30% NaOH solution containing NaCl, for 1 h at 0° C. The layers were again separated, and the aqueous phase extracted with t-BuOMe. The combined organic phase was washed with brine, dried over MgSO₄ and concentrated to yield 52.8 g as an amber oil.

Step 4. (2R, 3R)-2-hydroxy-3-methyl-5-phenylpentan-1-ol.

To a 5 L 3 neck round bottom flask equipped with a mechanical stirrer, thermocouple and nitrogen inlet was added hexanes (1 L) and cooled to 0° C. A 2.0 M solution of Me₃Al in hexanes (800 mL, 1.6 mol) was added, followed by a solution of the epoxide (120 g, 0.677 mol) in hexanes (250 mL)/CH₂Cl₂ (50 mL) maintaining the temperature below 20° C. Upon complete addition, the cloudy reaction mixture was stirred at 5° C. for 35 min, whereupon a solution of 10% HCl (300 mL) was added dropwise, followed by the addition of concd HCl (350 mL). The layers were separated, and the organic phase was washed with brine and dried over MgSO₄. After removal of the volatiles in vacuo, 122.1 gram of an oil was obtained.

Step 5. (2R, 3R)-2-hydroxy-3-methyl-5-phenylpent-1-yl Tosylate.

To a 2 L 3 neck round bottom flask equipped with a mechanical stirrer and nitrogen inlet was added the diol (58 g, 0.30 mol), dibutyltin oxide (1.5 g, 0.006 mol, 2 mol %), toluenesulfonyl chloride (57.5 g, 0.30 mol), CH₂Cl₂ (580 mL) and triethylamine (42.0 mL, 0.30 mol). The resulting mixture was stirred at room temperature for 2 h (although the reaction was complete within 1 h), filtered, washed with water and dried over MgSO₄. Concentration of the volatiles in vacuo afforded 104.1 gram of a slightly amber oil.

Step 6. (2R, 3R)-2-[(tert-Butyldimethylsilyl)oxyl-3-methyl-5-phenylpent-1-yl Tosylate.

A solution of the tosylate (100 g, 0.29 mol) and triethylamine (81.0 mL, 0.58 mol) in CH₂Cl₂ (1200 mL) was treated with neat TBS-OTf (99 mL, 0.43 mol) dropwise with continued stirring for another 20 min. The reaction was washed twice with brine, dried over MgSO₄ and concentrated to dryness. The oil was dissolved in a minimal amount of hexanes and filtered over a silica pad, eluting with hexanes:EtOAc (9:1) to yield a slightly amber oil, 134 g.

Step 7. (2R,3R,5RS) -2-[(tert-Butyldimethylsilyl)oxy]-3-methyl-5-bromo-5-phenylpent-1-yl Tosylate.

To a 5 L 3 neck round bottom flask equipped with a mechanical stirrer, reflux condenser and nitrogen inlet was added CCl₄ (1680 mL), TBS Ts (140 g, 0.30 mol), NBS (65 g, 0.365 mol) and AIBN (16.5 g, 0.10 mol). The mixture was degassed by evacuation under full vacuum with stirring, and backfilling with nitrogen (3×). The reaction mixture was then heated to reflux, whereupon the color became dark brown. After 15 min at vigorous reflux, the reaction mixture became light yellow, and chromatographic analysis indicated the reaction was complete. After cooling to room temperature, the reaction was filtered and the filtrate concentrated to dryness. The residue was redissolved in hexanes and filtered again, and concentrated to dryness to afford 170.3 gram as an amber oil.

Step 8. (2R, 3R)-2-[(tert-Butyldimethylsilyl)oxy]-3-methyl-5-phenylpent-4(E)-en-1-yl Tosylate.

To a 2 L 3 neck round bottom flask equipped with a mechanical stirrer, reflux condenser and nitrogen inlet was added a solution of the bromide (100 g, 0.186 mol) in acetonitrile (700 mL). DBU (83.6 mL, 0.557 mol) was added and the resulting dark brown solution was stirred at reflux for 15 min. After cooling to room temperature, the solvent was removed in vacuo, and the residue digested in CH₂Cl₂ (200 mL) and filtered through a silica pad. The volatiles were again evaporated, and the residue dissolved in EtOAc and washed with water, brine and dried over MgSO₄ and concentrated to dryness. Preparative mplc (Prep 500) chromatography afforded the desired unsaturated compound (50.3 g, 60% yield over 4 steps).

Step 9. (3S, 4R)-3-[(tert-Butyldimethylsilyl)oxy]-4-methyl-6-phenylhex-5(E)-en-1-nitrile.

The tosylate (50 g, 0.11 mol) was dissolved in DMSO (1 L) and treated with KCN (14.2 g, 0.22 mol) and water (25 mL), and the resulting mixture was stirred at 60° C. under nitrogen for 18 h. After cooling to room temperature, the reaction mixture was partitioned between EtOAc (1 L) and water (1 L). The aqueous phase was extracted with EtOAc (500 mL), and the combined organic phase was washed with brine and dried over Na₂SO₄. Flash chromatography over silica with CH₂Cl₂ afforded the desired nitrile in 92% yield.

Step 10. Methyl (5S, 6R)-5-[(tert-Butyldimethylsilyl)oxy]-6-methyl-8-phenylocta-2 (E),7(E)-dienoate.

The nitrile (14.67 g, 46.5 mmol) was dissolved in toluene (200 mL) and cooled to −78° C. under nitrogen. A 1.5M solution of DIBAL in toluene (37.2 mL, 55.8 mmol) was added dropwise with vigorous stirring. Upon complete addition, the cooling bath was removed and the reaction was stirred at room temperature for 1 h. The reaction mixture was carefully poured into 1N HCl and the mixture stirred at room temperature for 30 min. The layers were separated, and the organic phase was washed with a saturated aqueous solution of sodium potassium tartrate (2×), brine and dried over Na₂SO₄. The volatiles were removed in vacuo, and the crude pale yellow oil was used directly in the subsequent condensation. The crude aldehyde from above was dissolved in THF (90 mL) and treated with trimethyl phosphonoacetate (9.03 mL, 55.8 mmol) and tetramethylguanidine (7.0 mL, 55.8 mmol) at room temperature under nitrogen. The reaction mixture was stirred for 16 h, then partitioned between EtOAc (200 mL) and water (100 mL). The aqueous phase was back extracted with EtOAc (100 mL), and the combined organic phase was washed with water, brine and dried over Na₂SO₄. The volatiles were removed in vacuo, and the crude yellow oil (17.0 g) was chromatographed over silica gel with CH₂Cl₂:cyclohexane (1:1 to 2:1) to afford 13.67 grams of the desired ester, 78.5%.

### PREPARATIVE EXAMPLE 2

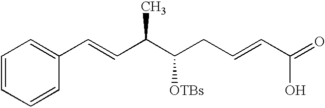

Methyl ester (2.673 mmol) was dissolved in acetone and then 1N aqueous LiOH (26 mL) added at room temperature. The cloudy mixture was further diluted with acetone (20 mL) and the resulting yellow mixture stirred at room temperature for 23.5 h. The reaction was diluted with diethylether (400 mL) and the organics washed with 1N HCl (120 mL), brine (200 mL) and H₂O (160 mL). The organics were dried and concentrated in vacuo to leave a yellow oil which was purified by column chromatography (gradient: 5% AcOH+20%-40% EtOAc/Hexanes ) to give carboxylic acid as a yellow oil (960 mg, 100%).

¹H NMR (CDCl₃) d 7.38-7.19 (m, PhH5), 7.09 (ddd, J=15.2, 7.6 and 7.9 Hz, 3-H), 6.38 (d, J=16 Hz, 8-H), 6.16 (dd, J=16 and 8 Hz, 7-H), 5.85 (d, J=15.8 Hz, 2-H),3.81-3.75 (m, 5-H), 2.49-2.37 (m, 6-H, 4-CH₂), 1.12 (d, J=6.7 Hz, 6-Me), 0.91 (s, SiCMe3), 0.065 (s, SiMe), 0.068 (s, SiMe) ppm;

IR u (CHC₁₃) 2957, 2930, 2858, 1697, 1258, 1098, 838 cm⁻¹;

MS (FD) 360.2 (M⁺, 100);

[a]_{D}+87.6° (c 10.5, CHCl₃);

Anal. calcd. for C₂₁H₃₂O₃ requires: C, 69.95; H, 8.95%.

Found: C, 69.19; H, 8.39%.

### PREPARATIVE EXAMPLE 3

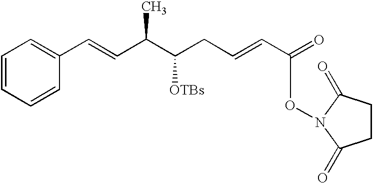

To a stirred solution of carboxylic acid (2 mmol) in dry dimethylformamide (5.50 mL) was added 1-ethyl-3-(3-dimethyaminopropyl)carbodiimide (2.4mmol) and N-hydroxysuccinimide (2.6 mmol) at room temperature. The mixture was stirred for 28 h and then diluted with EtOAc (100 mL) and washed with 1N aqueous HCl (2×50 mL), H₂O (75 mL), dried and concentrated in vacuo to leave an oil. Crude product was purified by column chromatography (gradient: 5-30% EtOAc/Hexanes) to give active ester as a pale yellow oil (724 mg, 80%).

¹H NMR (CDCl₃) d 7.36-7.20 (m, PhH₅, 3-H), 6.38 (d, J=16 Hz, 8-H), 6.14 (dd, J=16.1 and 8.0 Hz, 7-H). 6.03 (d, J=16 Hz, 2-H), 3.79 (q, J=4.3 Hz, 5-H), 2.94 (brs, CH₂CH₂), 2.58-2.42 (m, 6-H, 4-CH₂), 1.10 (d, J=6.8 Hz, 6-Me), 0.90 (s, SiCMe₃), 0.05 (s, SiMe₂) ppm;

IR u (CHCl3) 2957, 2931, 2858, 1772, 1741, 1648, 1364, 1254, 1092, 1069, 838 cm⁻¹;

MS (FD) 457 (M⁺, 100);

[a]D +71.30 (c 10.1, CHCl₃);

Anal. calcd. for C₂₅H₃₅NO₅ requires: C, 65.61; H, 7.71; N, 3.06%. Found: C, 65.51; H, 7.56; N, 3.02%.

### PREPARATIVE EXAMPLE 4

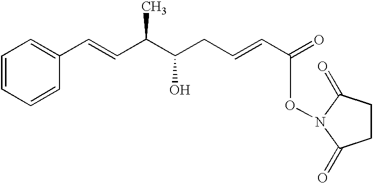

To a stirred solution of silyl ether (2.50 g, 5.47 mmol) in CH₃CN (130 mL) was added 48% aqueous HF (15 mL) at 0 C. The solution was stirred at 0 C. for 0.75 h and then at room temperature for 4 h. The reaction was diluted with diethylether (300 mL) and washed with H₂O until the wash was ˜pH7. Organics were dried (MgSO₄) and concentrated in vacuo to give a yellow residue which was recrystallized from Et2O to give alcohol as white crystals (1.46 g, 78%).

¹H NMR (CDCl₃) d 7.41-7.20 (m, PhH₅, 3-H), 6.48 (d, J=16 Hz, 8-H), 6.15-6.07 (m, 7-H, 2-H), 3.71-3.65 (m, 5-H), 2.83 (brs, CH₂CH₂), 2.60-2.33 (m, 6-H, 4-CH₂), 1.95 (brs, 5-OH), 1.14 (d, J=6.8 Hz, 6-Me) ppm;

IR u (KBr) 3457, 1804, 1773, 1735, 1724, 1209, 1099, 1067, 1049, 975, 744, 694 cm⁻¹;

UV (EtOH) lₘₐₓ 250 (e=20535) nm;

MS (FD) 343.2 (M⁺, 100);

[a]_{D} −57.8° (c 10.56, CHCl₃);

Anal. calcd. for C₁₉H₂₁NO₅S requires: C, 66.46; H, 6.16; N, 4.08%. Found: C, 66.49; H, 6.16; N, 4.07%.

### PREPARATIVE EXAMPLE 5

### (2S)-2-[3′-(tert-Butoxycarbonyl)amino-3′-(R)-benzylpropanoyloxy]-4-methylpentanoic Acid

(3R)-benzyl-3-aminopropanoic Acid (TFA Salt)

A sample of tert-Butyl 3-(R)-benzyl-3-aminopropanoate (Oxford Asymmetry, England, >99% e.e) was dissolved in trifluoroacetic acid (TFA) and then stirred at room temperature for 4 h. The trifluoroacetic acid was removed in vacuo to give an oily residue which was then triturated with methanol to give a white solid.

TLC: Rf=(CHCl3/CH3OH/NH4OH: 6:3.2:0.8)

¹HNMR(300 MHz, DMSO-d6) d: 7.93 (bs, 2H), 7.32 (m, 5H), 3.63 (t, J=7.2 Hz, 1H), 2.91 (dd, J=5.9 Hz, J=13.6 Hz, 2H), 2.77 (dd, J=8.1 Hz, J=13.6 Hz, 2H)

Anal: Calcd for C12H14NO4: C, 49.15; H, 4.81; N, 4.78. Found: C, 48.87; H, 4.73, N, 4.70.

N-(tert-butoxycarbonyl)-(3R)-benzyl-3-aminopropanoic Acid

A sample of (3R)-benzyl-3-aminopropanoic acid was dissolved in 1,4-dioxane/H2O/2.0 N NaOH (2:2:1) at 0° C. (ice bath). To this was then added di-t-butyl-dicarboxylate and the ice bath was removed and the resulting reaction mixture was let stirred at room temperature for 18 h. The reaction mixture was then concentrated to ca 10 ml and 25 ml of EtOAc was added. To this was then added 0.5 N NaHSO₄ to lower the pH of aqueous phase to ca. 2-3. The organic layer was then separated and the aqueous layer was extracted with EtOAc (20 ml×3). The combined EtOAc layer were then washed with water and brine and dried over NaSO4. The solvent was then removed in vacuo to give a pale yellow solid.

TLC: Rf=(CHCl3/CH3OH/NH4OH: 6:3.2:0.8)

IR (cm⁻¹): 3361, 2985, 1670, 1686, 1526, 1266, 1168, 700.

UV (CH₃OH): 258 nm (e=158).

1HNMR(300 MHz, DMSO-d6) d: 7.20 (m, 5H), 6.75 (d, J=8.6 Hz, 1H), 3.88 (m, 1H), 2.64 (d, J=7.0 Hz, 2H), 2.28 (t, J=5.1 Hz, 2H)1.27 (s, 9H).

Mass (FAB): 280 (M⁺+H).

Allyl (2S)-2-[3′-N-(tert-Butoxycarbonyl)amino-3′-(R)-benzylpropanoyloxy]-4-methylpentanoate

To a solution of allyl (2S)-2-hydroxy-4-methylpentanoate and (3R)-benzyl-3-(tert-butoxycarbonyl)aminopropanoic acid in 10 ml of dry methylene chloride at 0° C. (ice bath), was added dicyclohexylcarbodiimide and then followed by DMAP. The reaction mixture was then stirred at room temperature for 3 hours. The reaction mixture was then filtered through a small pad of celite and the filtrate was washed with 5% NaHCO₃, brine and dried over Na₂SO₄. The solvent was removed in vacuo and the residue was flash chromatographed on SiO₂ (15% EtOAc/hexane) to give a clear oil.

IR (cm⁻¹): 2961, 2933, 1742, 1715, 1497, 1366, 1249, 1170, 1127.

UV (CH₃OH): 258 nm (e=218).

1HNMR (300 MHz, CDCl3) d: 7.25 (m, 5H), 5.89 (m, 1H), 5.20-5.36 (m, 3H), 5.10 (dd, J=3.9 Hz, J=9.6 Hz, 1H), 4.65 (d, J=5.4 Hz, 2H), 4.15 (bs, 1H), 2.87 (m, 2H), 2.62 (dd, J=5.6 Hz, J=15.4 Hz, 1H), 2.50 (dd, J=5.0 Hz, J=15.4 Hz, 1H), 1.60-1.85 (m, 3H), 1.40 (s, 9H), 0.95 (d, J=4.3 Hz, 3H), 0.93 (d, J=4.3 Hz, 3H).

Mass(FAB): 434.4 (M⁺+H).

Anal: Calcd for C24H35NO6: C, 66.49; H, 8.14; N, 3.23. Found: C, 66.32; H, 8.29, N, 3.42.

Carboxylic Acid Deprotection

To a sample of 0.98 g (2.26 mmol) of allyl (2S)-2-[3′-N-(tert-Butoxycarbonyl)amino-3′-(R)-benzylpropanoyloxy]-4-methylpentanoate, 0.277 g (0.23 mmol) of tetrakistriphenylpalladium in 50 ml of dry THF was added 2.2 ml of anhydrous morpholine. The reaction mixture was then let stirred at room temperature for 1.5 h and TLC showed the disappearance of the starting material. The reaction mixture was then diluted with 30 ml of diethyl ether and washed with 50 ml of 1.0 N HCl (×2). The organic layer was then extracted with 2×50 ml of 5% NaHCO3. The combined aqueous layer was then acidified with 0.5-1.0 N HCl to pH 3-4 and then extracted with ether (400 mL). The ether layer was then washed with brine and dried over Na2SO4 and concentrated in vacuo to give 0.73 g (82%) of the title compound as a pale yellow solid.

IR (cm-¹): 3034, 2962, 2934, 1727, 1709, 1498, 1455, 1393, 1369, 1253, 1198, 1165, 1127.

UV (CH30H): 259 nm (e=214).

¹HNMR(300 MHz, CDCl3) d: 7.20 (m 5H), 5.13 (m, 2H), 4.19 (bs, 1H), 2.84 (m, 2H), 2.40-2.65 (m, 2H),1.60-1.85 (m, 3H), 1.38 (s, 9H), 1.23 (d, J=6.8 Hz, 3 H), 0.96 (d, J=5.8 Hz, 3H), 0.93 (d, J=5.8 Hz, 3H).

Mass(FD): 394.4 (M⁺+H).

Anal: Calcd for C21H31NO6: C, 64.10; H, 7.94; N, 3.56. Found: C, 64.16; H, 7.97, N, 3.43.

### PREPARATIVE EXAMPLE 6

### (2S)-2-[3′(tert-Butoxycarbonyl)amino-3′-(R)-methylpropanoyloxy]-4-methylpentanoic Acid

(3R)-methyl-3-aminopropanoic Acid (TFA Salt)

A sample of 750 mg (4.7 mmol) of tert-Butyl 3-(R)-methyl-3-aminopropanoate (Oxford Asymmetry, England, >99% e.e) was dissolved in 7.0 ml of trifluoroacetic acid (TFA) and then stirred at room temperature for 4 hours. The trifluoroacetic acid was removed in vacuo to give an oily residue which was then triturated with methanol to give a white solid, yield: 1.05 g (100%)

TLC: Rf=0.15 (CHC13/CH3OH/NH4OH: 6:3.2:0.8)

IR (cm⁻¹): 3286, 3092, 2996, 2914, 1714, 1654, 1504, 1448, 1237, 1196, 1143, 723.

¹HNMR(300 MHz, CD3OD) d: 3.61 (q, J=6.6 Hz, 1H), 2.62 (t, J=6.0 Hz, 2H), 1.32 (d, J=6.7 Hz, 3H)

Anal: Calcd for C6H10NO4F3: C, 33.19; H, 4.64; N, 6.45. Found: C, 33.32; H, 4.64, N, 6.46.

N-(tert-butoxycarbonyl) (3R)-methyl-3-aminopropanoic acid

A sample of 1.0 g (4.6 mmol) of (3R)-methyl-3-aminopropanoic acid was dissolved in 20 ml of 1,4-dioxane/H₂O/2.0 N NaOH (2:2:1) at 0° C. (ice bath). To this was then added 1.16 ml (5.06 mmol) of di-t-butyl-dicarboxylate and the ice bath was removed and the resulting reaction mixture was stirred at room temperature for 18 hours. The reaction mixture was then concentrated to 10 ml and 25 ml of EtOAc was added. To this was then added 0.5 N NaHSO₄ to lower the pH of aqueous phase to 2-3. The organic layer was then separated and the aqueous layer was extracted with EtOAc (20 ml×3). The combined EtOAc layer were then washed with water and brine and dried over NaSO₄. The solvent was then removed in vacuo to give a pale yellow solid. Yield: 0.88 g (94%).

TLC: Rf=0.52 (CHCl₃/CH₃OH/NH₄OH: 6:3.2:0.8)

IR (cm⁻¹): 2981, 1711, 1504, 1368, 1244, 1166.

¹HNMR (300 MHz, DMSO-d6) d:3.74 (m, 1H), 2.35 (dd, J=6.3 Hz, J=15.2 Hz, 1H), 2.16 (dd, J=7.6 Hz, J=15.2 Hz, 1H)1.33 (s, 9H), 0.99 (d, J=6.6 Hz, 3H).

Mass(FAB): 204.2 (M⁺+H), 223.1 (M⁺+Na).

Anal: Calcd for C9H17NO4: C, 53.19; H, 8.43; N, 6.89. Found: C, 53.42; H, 8.69, N, 6.77.

Allyl (2S)-2-[3′-N-(tert-Butoxycarbonyl)amino-3′-(R)-methylpropanoyloxy]-4-methylpentanoate

To a solution of 0.81 g (3.98 mmol) of allyl (2S)-2-hydroxy-4-methylpentanoate (1) and 0.82 g (4.78 mmol, 1.2 eq) of N-[tert-butoxycarbonyl] (3R)-methyl-3-aminopropanoic acid in 10 ml of dry methylenechloride at 0° C. (ice bath), was added 0.91 g (4.4 mmol) of dicyclohexylcarbodiimide and then followed by 0.13 g (0.96 mmol, 0.2 eq) of DMAP. The reaction mixture was then let stirred at room temperature for 3 h (TLC indicated the completion of the reaction). The reaction mixture was then filtered through a small pad of celite and the filtrate was washed with 5% NaHCO₃, brine and dried over Na2SO4. The solvent was removed in vacuo and the residue was flash chromatographed on SiO2 (15 % EtOAc/hexane) to give 1.3 g (91%) of (2) as a clear oil.

TLC: Rf=0.48 (20% EtOAc/hexane)

IR (cm⁻¹): 3442, 2963, 2937. 2874, 1738, 1706, 1503, 1469, 1456, 1391, 1368, 1341, 1274, 1239, 1169, 1121, 1104, 1013, 112, 930.

¹HNMR (300 MHz, DMSO-d6) d: 6,76 (d, J=7.7 Hz, 1H), 5.84 (m, 1H), 5.26 (d, J=17.5 Hz, 1H), 5.18 (d, J=10.4 Hz, 1H), 4.89 (dd, J=4.0 Hz, J=9.0 Hz, 1H), 4.56 (d, J=4.9 Hz, 2H), 3.77 (m, 1H), 2.55 (dd, J=6.2 Hz, J=15 Hz, 1H), 2.31 (dd, J=7.9 Hz, J=15 Hz, 1H), 1.69 (m, 2H), 1.54 (m, 1H), 1.33 (s, 9H), 1.01 (d, J=6.6 Hz, 3H), 0.87 (d, J=6.2 Hz, 3H), 0.84 (d, J=6.3 Hz, 3H).

Mass(FAB): 358.2 (M⁺+H).

Anal: Calcd for C18H31NO6: C, 60.48; H, 8.74; N, 3.92. Found: C, 60.50; H, 8.96, N, 3.66.

### PREPARATIVE EXAMPLE 7

### Synthesis of (2)

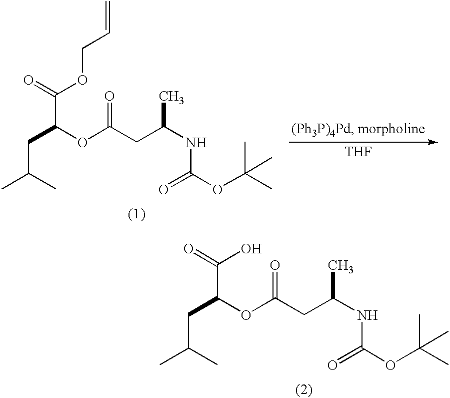

To a sample of 1.23 g (3.44 mmol) of (1), 0.40 g (0.344 mmol) of tetrakistriphenyl-palladium in 70 ml of dry THF was added 3.31 ml of anhydrous morpholine. The reaction mixture was then let stirred at room temperature for 1.5 h and TLC showed the disappearance of the starting material. The reaction mixture was then diluted with 30 ml of diethyl ether and washed with 200 ml of 1.0 N HCl. The organic layer was then extracted with 3×200 ml of 5% NaHCO3. The combined aqueous layer was then acidified with 0.5-1.0 N HCl to pH 3-4 and then extracted with ether (400 mL). The ether layer was then washed with brine and dried over Na2SO4 and concentrated in vacuo to give a pale yellow solid (2) weight 1.02 g (93%).

IR (cm⁻¹): 2980, 2963, 2934, 2873, 1727, 1504, 1456, 1411, 1392, 1369, 1342, 1245, 1168, 1128, 1104, 1065.

¹HNMR (300 MHz, DMSO-d6) d: 5.09 (m, 1H), 5.01 (m, 1H), 4.08 (m, 1H), 2.61 (m, 2H), 1.74 (m, 3H), 1.44 (s, 9H), 1.23 (d, J=6.8 Hz, 3 H), 0.96 (d, J=6.2 Hz, 3H), 0.93 (d, J=6.2 Hz, 3H).

Mass(FAB): 318.2 (M⁺+H), 340.2 (M⁺+Na).

Anal: Calcd for C15H27NO6: C, 56.77; H, 8.57; N, 4.41. Found: C, 57.82; H, 9.08, N, 4.11.

### Preparative Example 8

### N-tert-butoxycarbonyl 1-(aminomethyl)cyclopropane-1-carboxylic Acid

Reduction

To a 500 ml Parr hydrogenator bottle were charged 3.0 g (27 mmol) of 1-cyano-1-cyclopropanecarboxylic acid (Aldrich) and 1.0 g of platinum (IV) oxide in 250 mL of glacial acetic acid. The mixture was hydrogenated at 60 psi hydrogen for 4 h. After filtering away the catalyst, the volatiles were removed in vacuo and the solid was dried in a vacuum oven at 75° C. This solid was then triturated in CHCl₃, filtered and dried to give 2.7 g (86%) of 1-aminomethyl-1-cyclopro-panecarboxylic acid as a white solid.

m.p.=261-262° C. (foam, dec)

Mass (FD) M+1=116

Nitrogen Protection

To a 250 mL 24/40 round bottom flask were charged 1.5 g (13.0 mmol) of 1-aminomethyl-1-cyclopropanecarboxylic acid dissolved in 28 mL of 1,4-dioxane, 15 mL water, and 2N NaOH. The solution was then cooled down in an ice bath, followed by the slow addition of 3.3 mL (14.3 mmol) of di-t-butyl dicarbonate. The reaction was stirred at room temperature for 21 h. The 1,4-dioxane was removed in vacuo and the aqueous was diluted with additional water and layered with EtOAc. The pH of the stirring solution was adjusted to 3 using 0.5 N NaHSO₄. The organic layer was separated away, and the aqueous was extracted with EtOAc. The organic layers were combined, washed with brine, dried, over Na₂SO₄, filtered and removed in vacuo to give 2.6 g (93%) of the title compound as a white solid.

m.p.=104-106° C.

MASS (FD) M+1=216

Anal: Calcd for C15H21NO4: C, 64.50; H, 7.58; N, 5.01. Found: C, 63.25; H, 7.35, N, 4.99.

### Preparative Example 9

### Preparation of N-[tert-butoxycarbonyl] O-[1-carboxy-3-methylbut-1-yl] 1-(aminomethyl)cyclopropane-1-carboxylate

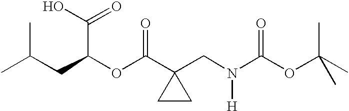

Coupling

A solution of 2.5 gm (11.88 Mol) N-tert-butoxy-carbonyl 1-aminomethylcyclopropanecarboxylic acid, 1.86 gm (10.8 mMol) allyl 2-hydroxy-4-methylpentanoate, and 0.28 gm (2.3 mmol) dimethylaminopyridine in 25 mL dichloromethane was cooled in ice bath as a solution of 2.58 gm (13.07 mmol) DCC in 12 mL dichloromethane was added slowly. After stirring at room temperature for 4 hours, the reaction mixture was filtered and the filtrate washed sequentially with saturated aqueous sodium bicarbonate (three times) and saturated aqueous sodium chloride. The remaining organic phase was dried over sodium sulfate and concentrated under reduced pressure. The residual oil was subjected to flash silica gel chromatography, eluting with hexane containing 15% ethyl acetate. Fractions containing product were combined and concentrated under reduced pressure to provide 2.8 gm (70%) of N-[tert-butoxycarbonyl] O-[1-(allyloxycarbonyl-3-methylbut-1-yl] 1-aminomethylcyclopropanecarboxylate as a colorless oil.

MS(FD): m/e=370 (M+1)

EA: Calculated for: C₁₉H₃₁NO₆: C, 61.77; H, 8.46; N, 3.79. Found: C, 61.50; H, 8.25; N, 3.69.

Deprotection

Beginning with 2.7 gm (7.31 mMol) of N-[tert-butoxycarbonyl] 1-(allyloxycar-bonyl-3-methylbut-1-yl] 1-aminomethylcyclopropanecarboxylate, 2.3 gm (95%) of the title compound were prepared essentially as described in Preparative Example 7.

m.p. =90-92° C.

MS(FD): m/e=330 (M+1)

EA: Calculated for: C₁₆H₂₇NO₆: C, 58.34; H, 8.26; N, 4.25. Found: C, 58.37; H, 8.07; N, 4.14.

### PREPARATIVE EXAMPLE 10

### N-[tert-butoxycarbonyl] 1-(aminomethyl)cyclopentane-1-carboxylic Acid

Ethyl 1-(cyano)cyclopentane-1-carboxylate

A suspension of 6.5 gm (200 mMol) cesium carbonate in 250 mL dimethylformamide containing 11.9 mL (100 mMol) 1,4-dibromobutane was cooled to 5° C. in an ice bath. A solution of 10.7 mL (100 mMol) ethyl cyanoacetate in 100 mL dimethylformamide was added dropwise over 25 minutes. The cooling bath was removed and the reaction mixture was stirred for 6 hours at room temperature. The reaction mixture was partitioned between hexane and water. The hexane phase was dried over magnesium sulfate and concentrated under reduced pressure. The residue was partitioned between methyl tert-butyl ether and water. The aqueous phase was washed with methyl tert-butyl ether and these organic phases are combined, washed with 3×100 mL 5% aqueous lithium chloride, dried over magnesium sulfate and concentrated under reduced pressure. The residue was distilled under vacuum (11 mm Hg) through a Vigreaux column to provide 13.75 gm (82%) of the desired compound.

b.p.=108-110° C.

MS(FD): m/e=168 (M+1)

EA: Calculated for: C₉H₁₃NO₂: C, 64.65; H, 7.84; N, 8.38. Found: C, 64.53; H, 7.97; N, 8.56.

1-aminomethyl-1-hydroxymethylcyclopentane

A solution of 13.27 gm (79.4 mMol) ethyl 1-(cyano)-cyclopentane-1-carboxylate in 150 mL tetrahydrofuran was cooled to −70° C. and then 168 mL (168 mMol) lithium aluminum hydride bis(tetrahydrofuran) solvate (1 M in toluene) was added dropwise over 30 minutes. The reaction mixture was stirred at −78° C for 3 hours and was then allowed to warm gradually to room temperature. After an additional 15 hours the reaction mixture was treated sequentially with 6.4 mL water, 6.4 mL 15% sodium hydroxide, and then 19 mL water. After stirring for 20 minutes the mixture is treated with HYFLO™ and filtered through a bed of HYFLO™. The filter cake was washed with 3×50 mL tetrahydrofuran and the filtrate was concentrated under reduced pressure. The residual oil was distilled in a Kugelrohr apparatus to provide 8.93 gm of the desired compound as a clear oil. b.p.=110-125° C. (11 mm Hg)

N-[tert-butoxycarbonyl] 1-aminomethyl-1-hydroxymethylcyclopentane

A solution of 8.7 gm (67.4 mMol) 1-aminomethyl-1-hydroxymethylcyclopentane in 120 mL tetrahydrofuran was treated with 69 mL 1N sodium hydroxide followed by 16.2 gm (74.2 mMol) di-(tert-butyl) dicarbonate and the mixture was stirred at room temperature. After 17 hours the phases were separated and the aqueous phase filtered. The filtrate was extracted with 200 mL methyl tert-butyl ether and the organic phases combined, washed with 200 mL saturated aqueous sodium chloride, dried over magnesium sulfate and concentrated under reduced pressure. The residue was slurried in 150 mL hexane for 1 hour, filtered, and dried at 35° C. for 3 hours to provide 13.04 gm (85%) of the desired compound as a white solid.

Oxidation

A solution of 12.47 gm (54.4 mMol) N-[tert-butoxycarbonyl] 1-aminomethyl-1-hydroxymethylcyclopentane in 300 mL acetonitrile and 150 mL carbon tetrachloride was treated with a solution of 46.6 gm (218 mMol) sodium periodate in 400 mL water followed by 0.224 gm (1.1 mMol) ruthenium(III) chloride monohydrate. The reaction mixture was stirred for 7 hours at room temperature and was then filtered. The filtrate was diluted with 250 mL dichloromethane and the phases separated. The aqueous phase was extracted with 200 mL dichloromethane. The combined organic phases were extracted with 2×200 mL and then 100 mL of 1N sodium hydroxide. The combined basic extracts were stirred with 300 mL dichloromethane and then 500 mL 1M aqueous sodium hydrogensulfate was added dropwise. The aqueous phase was washed with 2×200 mL dichloromethane and the combined organic phases were filtered through HYFLO™. The filtrate was washed with 250 mL saturated aqueous sodium chloride, dried over magnesium sulfate, filtered through HYFLO™, and concentrated under reduced pressure. The residue was dissolved in 300 mL methyl tert-butyl ether and 100 mL dichloromethane, and was then treated with DARCO™. The mixture was filtered through HYFLO™ and concentrated under reduced pressure to provide 10.56 of the title compound as gray crystals.

### PREPARATIVE EXAMPLE 11

### Preparation of N-[tert-butoxycarbonyl] O-[1-carboxy-3-methylbut-1-yl] 1-aminomethyl-cyclopentanecarboxylate

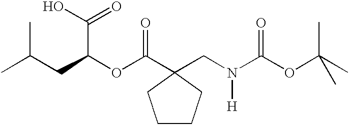

Beginning with N-[tert-butoxycarbonyl] 1-(aminomethyl)cyclopentane-1-carboxylic acid, the title compound was prepared essentially as described in Preparative Example 9.

[α]_{D}(CHCl₃)=−18.3° (c=10 mg/mL)

EA: Calculated for: C₁₈H₃₁NO₆: C, 60.48; H, 8.74; N, 3.92. Found: C, 60.54; H, 8.82; N, 3.95.

### PREPARATIVE EXAMPLE 12

### (2S)-2-[3′(tert-Butoxycarbonyl)amino-5′-methylhexanoyloxyl]-4-methylpentanoic Acid

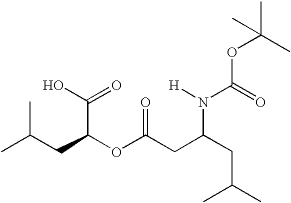

Beginning with tert-butyl 3(R)-3-amino-5-methylhexanoate, the title compound was prepared essentially as described in Preparative Example 5.

MS(FD): m/e=360 (M+1)

HRMS: Calculated for C₁₈H₃₃NO₆: 360.2386. Found: 360.2392.

### PREPARATIVE EXAMPLE 13

### (2S)-2-[3′(tert-Butoxycarbonyl)amino-3′-phenylpropanoyloxy]-4-methylpentanoic acid

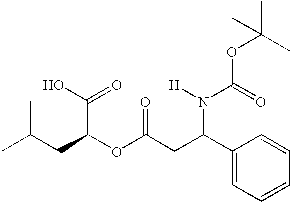

Beginning with tert-butyl 3(S)-3-amino-3-phenylpropanoate, the title compound was prepared essentially as described in Preparative Example 5.

m.p.=97-99° C.

MS(FD): m/e=380 (M+1)

EA: Calculated for: C₂₀H₂₉NO₆: C, 63.30; H, 7.70; N, 3.69. Found: C, 63.38; H, 7.45; N, 3.75.

### EXAMPLE 1

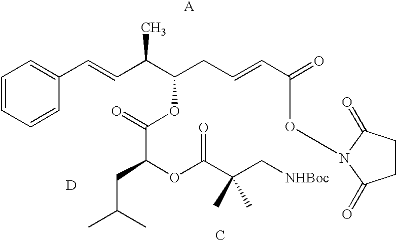

To a suspension of carboxylic acid (1.28 g, 3.87 mmol), in dry dichloromethane (6 mL) was added EDC (742 mg, 3.87 mmol) and DMAP (73 mg, 0.60 mmol) and the mixture stirred at room temperature for 0.5 h. A solution of alcohol (1.02 g, 2.97 mmol) in dichlormethane (5.5 mL) was added to the reaction mixture and stirred for a further 0.3 h. The reaction was diluted with CH₂Cl₂ (200 mL) and washed with 1N aq. HCl (2×50 mL), sat. aq. NaHCO₃ (2×50 mL), H₂O (50 mL). The organics were dried (MgSO₄) and concentrated in vacuo to leave an oily residue, which was purified by column chromatography (gradient: 10-30% EtOAc/Hexanes) to give the desired ester as a yellow solid (1.68 g, 79%).

1H NMR (CDCl3) unit A d 7.35-7.20 (m, PhH5,3-H), 6.43 (d, J=15.8 Hz, 8-H), 6.12 (d, J=15.9 Hz, 2H), 5.99 (dd, J=8.5 and 15.8 Hz, 7-H), 5.06-5.08 (m, 5-H), 2.85 (brs, CH₂CH₂), 2.68-2.61 (m, 6-H, 4-CH₂), 1.13 (d, J=6.8 Hz, 6-Me); unit C d 5.31 (brt, NH), 3.28-3.25 (m, 3-CH₂), 1.43 (s, CMe₃), 1.21 (s, 2Me), 1.19 (s, 2-Me); unit D d 4.95 (dd, J=9.8 and 3.8 Hz, 2-H), 1.73-1.64 (m, 3-H, 4-H), 1.59-1.49 (m, 3-H′), 0.85 (d, J=6.4 Hz, 5-Me), 0.82 (d, J=6.4, 4-Me) ppm;

IR u (KBr) 3400, 2975, 1743, 1367, 1206, 1126, 1145, 1068 cm ⁻¹;

MS (FD) 657 (M⁺, 100);

[a]_{D}+39.5° (c 10.38, CHCl₃);

Anal. calcd. for C₃₅H₄₈N₂O₁₀ requires: C, 64.01; H, 7.37; N, 4.27%. Found: C, 64.19;H, 7.27; N, 4.52%.

### EXAMPLE 2

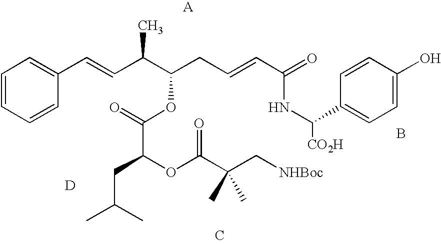

To a stirred solution of active ester (150 mg, 0.229 mmol) in dry DMF (2.5 mL) was added N,O-Bis-(trimethylsilyl)acetamide (282 uL, 1.143 mmol) followed by D-Hydroxy-phenylglycine (57 mg, 0.343 mmol). The mixture was heated in a sealed tube under N₂ at 55 C. for 20 h. Reaction solution was diluted with EtOAc (180 mL) and washed with 1N aq. HCl (50 mL), H₂O (50 mL), brine (50 mL), dried (MgSO₄) and concentrated in vacuo to give a yellow solid. Purification of the crude solid by column chromatography (gradient: 5-20% MeOH/CH₂Cl₂) provided amide (122 mg, 75%).

¹H NMR (CD3OD/CDCl₃) Unit A d 7.27-7.20 (m, PhH5), 6.75-6.69 (m, 3-H), 6.43 (d, J=15.9 Hz, 8-H), 5.96 (d, J=15.7 Hz, 7-H), 5.93 (d, J=15.6 Hz, 2-H), 4.95-4.93 (m, 5-H), 2.56-2.49 (m, 6-H, 4-CH₂), 1.04 (d, J=6.8 Hz, 6-Me); Unit B d 7.16 (d, J=8.3 Hz, ArH₂), 6.66 (d, J=8.2 Hz, ArH₂), 5.62 (brt, NH)5.19-5.18 (m, 2-H); Unit C d 3.15 (d, J=6.3 Hz, 3-CH₂), 1.36 (s, CMe₃), 1.11 (s, 2-Me), 1.08 (s, 2-Me); Unit D d 4.85 (dd, J=9.6 and 3.3 Hz, 2-H), 1.64-1.57 (m, 3-H, 4-H), 1.55-1.47 (m, 3-H′), 0.76 (d, J=6.3 Hz, 5-Me), 0.73 (d, J=6.3 Hz, 4-Me) ppm

IR u (KBr) 3400, 2972, 1728, 1672, 1614, 1515, 1450, 1416, 1171, 1147 cm⁻¹;

MS (FAB) 610.6 ([MH₂-Boc]⁺, 100);

[a]_{D} −19.9° (c 6.53, MeOH).

### EXAMPLE 3

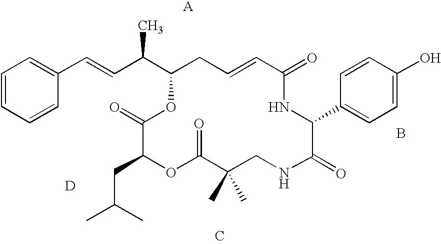

Boc amine as prepared by Example 2 (109 mg, 0.154 mmol) was dissolved in trfluoroacetic acid (5 mL, 5 mM) and stirred at room temperature for 2 h. The reaction was concentrated in vacuo and dried under high vacuum to give the trifluoroacetate salt of amine as a light brown foam. Crude amine salt (max. 0.154 mmol) was dissolved in dry DMF (31 mL) and diisopropylethylamine (80 uL, 0.462 mmol), followed by pentafluorophenyl diphenyl-phosphinate (77 mg, 0.2 mmol) added. The resulting solution was stirred at room temperature under dry N₂ for 15 h, concentrated in vacuo and the residue purified by column chromatography (gradient: 1-4% MeOH/CH₂Cl₂) to provide cryptophycin as a tan solid (54 mg, 59%).

¹H NMR (CDCl₃) Unit A d 7.36-7.15 (m, PhH₅), 6.79-6.69 (m, 3-H), 6.54 (d, J=15.8,8-H), 5.98 (dd, J=15.8 and 8.8 Hz, 7-H), 5.06-5.0 (m, 5-H), 2.61-2.49 (m, 6-H, 4-H), 2.39-2.30 (m, 3-H′), 1.10 (d, J=6.8 Hz, 6-Me); Unit B d 7.90 (dd, J=10 and 1.68 Hz, OH), 7.65 (d, J=6.3 Hz, NH), 7.10 (d, J=8.5, ArH₂), 6.71 (d, J=8.4,ArH₂), 5.28 (d, J=6.5 Hz, 2-H), ; Unit C d 3.55-3.47 (dd, J=13.3 and 10.1 Hz, 3-CH₂), 3.00 (d, J=13.4 Hz, NH) 1.19 (s, 2-Me), 1.16 (s, 2-Me); Unit D d 4.90 (dd, J=10 and 3.5 Hz, 2-H), 1.66-1.54 (m, 3-H, 4-H), 1.32-1.25 (m, 3-H′), 0.67 (apparent t, J=7.1 Hz, 5-Me, 4-Me) ppm;

IR u (KBr) 3418, 3340, 2960, 1740, 1713, 1671, 1514, 1271, 1198, 1155, 972 cm⁻¹;

MS (FD) 590 (M⁺, 100);

[a]_{D}+15.35° (c 3.91, CHCl₃).

### EXAMPLE 4

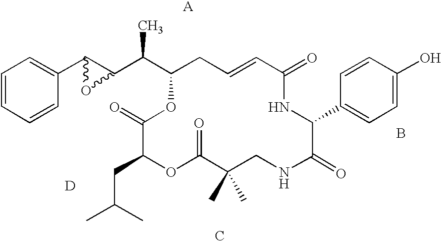

Styrene prepared as described by Example 3 (42 mg, 0.0712 mmol) was suspended in dry dichloromethane (2.2 mL, 0.035 mM) and mCPBA (49 mg, 0.285 mmol) added in one portion at room temperature. Dry tetrahydrofuran (0.3 mL) was added to produce a homogeneous solution. The reaction was stirred under N₂ at room temperture for 21 h and then diluted with further CH₂C₁₂ (15 mL). Organics were washed with 10% aq. Na₂S₂O₅ (10 mL), sat. aq. NaHCO₃ (10 mL), H₂O (10 mL), dried (MgSO₄) and concentrated in vacuo to give a yellow solid. Crude product was initially purified by column chromatography (gradient: 1-5% MeOH/CH₂Cl₂) to give a 1:1.15 mixture of a:b C7-C8 epoxides as a white solid (23 mg, 54%).Reverse phase HPLC (column: 4.6×250 mm Kromsil C18; Eluent: 60% CH₃CN/H₂O; Flow: 1.0 mL/min; UV: 220nm) separation of the a:b mixture provided a-epoxide (2.3 mg, t=13.7min) and b-epoxide (5.8 mg, t=12.1 min) as white solids.

### EXAMPLE 5

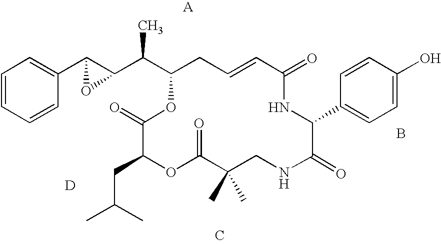

The above illustrated compound was prepared substantially as described above using the procedures of Examples 1-4

α-Epoxide:

¹H NMR (CDCl₃)

### EXAMPLE 6

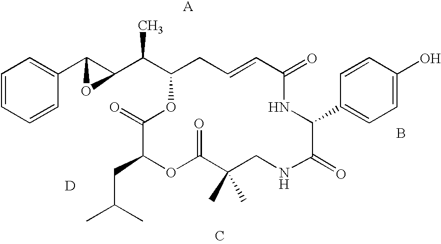

The above illustrated compound was prepared substantially as described above using the procedures of Examples 1-4

β-Epoxide:

¹H NMR (CDCl₃) Unit A d 7.36-716 (m, PhH₅), 6.70-6.79 (m-H), 5.91 (dd, J=15.5 and 5.18 Hz, 2-H) 5.23-5.18 (m, 5-H), 3.75 (d, J=1.67 Hz, 8-H), 2.96 (dd, J=7.4 and 2.0 Hz, 7-H), 2.72-2.67 (m, 4-H), 2.44-2.39 (m, 4-H′), 1.81-1.88 (m, 6-H), 1.13 (d, J=6.9,6-Me); Unit B d 7.66 (s, NH), 7.13 (d, J=8.5 Hz, ArH₂), 6.74 (d, J=8.5 Hz, ArH₂), 5.27 (s, 2-H); Unit C d 7.66 (s, NH), 3.49 (dd, J=13.6 and 10 Hz, 3-CH₂), 1.20 (s, 2-Me), 1.18 (s, 2-Me); Unit D d 4.93 (dd, J=10 and 3.2 Hz, 2-H), 1.69-1.59 (m, 3-H, 4-H), 1.30-1.22 (m, 3-H′), 0.79 (d, J=6.2 Hz, 5-Me), 0.78 (d, J=6.3 Hz, 4-Me) ppm.

### EXAMPLE 7

### Synthesis of Compound (M)

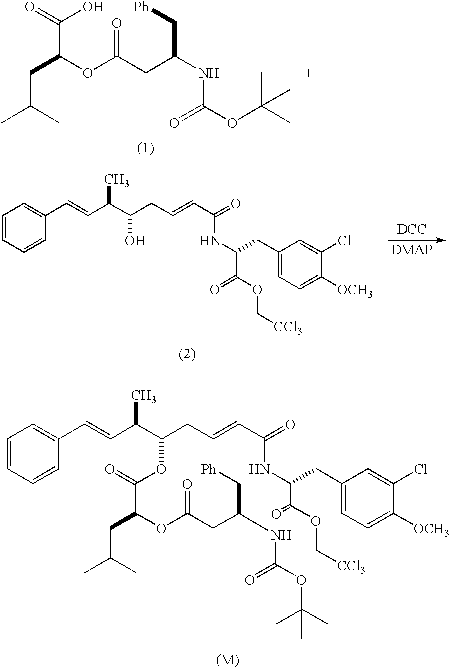

To a flame-dried 100 ml three-neck round bottom flask under argon was added 0.66 g (1.68 mmol, 1.5 eq) of (2S)-2-[3′(tert-Butoxycarbonyl) amino-2′-(R)-benzylpropanoyloxy]-4-methylpentanoic acid (1) and 0.66 g (1.13 mmol ) of compound (2) (Barrow, R. A. et al., J. Am. Chem. Soc. 117, 2479-2490 (1995)) in 15 ml of dry methylene chloride at 0° C. To this solution was then added 34 mg of DMAP and 0.37 g (1.68 mmol, 1.50 eq) of dicyclohexylcarbodiimide and the resulting solution was let stirred at RT for 18 h. TLC showed the completion of the reaction after 18 h and the white precipitate was filtered through a short pad of celite and the filtrate was diluted with 500 ml of ether. The organic layer was then washed with 50 ml of 1.0 N HCl followed by 50 ml of 5% NaHCO₃. The solvent was then removed in vacuo to give a crude solid which weight ca. 1.22 g. This crude solid was then flash chromatographed on SiO2 (35:65 EtOAc/hexane) to give a total of 0.958 g (88%) of coupled product (M) as a foamed white solid.

IR (cm⁻¹): 1734, 1706, 1679, 1503, 1281, 1259, 1169.

UV (95% EtOH): 230 nm (e=21,823), 246 nm (e=21,462).

¹HNMR (300 MHz, DMSO-d6) d: 7.16-7.34 (m, 11H), 7.04 (dd, J=2.0 Hz, J=8.4 Hz, 1H), 6.79 (d, J=8.2 Hz, 1H), 6.74 (m, 1H), 6.55 (d, J=8 Hz, 1H), 6.39 (d, J=15.9hz, 1H), 5.96 (dd, J=8.6 Hz, J=15.8 Hz, 1H), 5.89 (d, J=15.8 Hz, 1H), 5.04 (m, 3H), 4.93 (dd, J=3.8 Hz, J=8.0 Hz, 1H), 4.76 (d, J=11.9 Hz, 1H), 4.65 (d, J=11.9 Hz, 1H), 4.12 (m, 1H), 3.82 (S, 3H), 3.15 (m, 1H), 3.08 (m, 1H), 2.85 (m, 2H),2.50 (m, 5H), 1.50-1.75 (m, 3H), 1.38 (s, 9H), 1.10 (d, J=6.8 Hz, 3H), 0.85 (d, J=6.4 Hz, 3H), 0.79 (d, J=6.4 hz, 3H).

Mass(FAB): 965.4 (M⁺+H).

Anal: Calcd for C48H58N2O10C14: C, 59.76; H, 6.06; N, 2.90. Found: C, 59.74; H, 6.19, N, 2.97.

### EXAMPLE 8

### Synthesis of Compound (N)

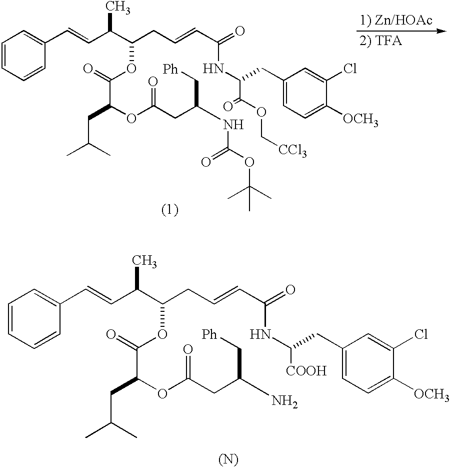

A sample of 0.80 g (0.83 mmol) of (1) was dissolved in 30 ml of glacial acetic acid and to this was added 3.0 g of zinc dust. The mixture was then sonicated (room temperature) for 1.5 h (TLC showed the complete consumption of the starting material). The zinc dust was then filtered away through a short pad of celite and the filtrate was concentrated to give a white solid. This crude solid was then dissolved immediately in 7.5 ml of trifluoroacetic acid and let stirred at room temperature for 2 h. TFA was then removed in vacuo and the oily solid was triturated with ether/hexane to give a white solid (0.814 g). This crude solid looks very good by TLC and 1HNMR, and this was then dissolved in ca. 50 ml of distilled water and triturated for 1 h (with sonication). The white solid was then collected and dried in vacuo at 50° C. to give 0.55 g (78% in two steps) of TFA salt of compound (N) (the aqueous filtrate from water trituration, however, did not contain any UV active material).

IR (cm⁻¹): 2964, 1729, 1674, 1628, 1501, 1397, 1280, 1258, 1148, 1088.

UV (95%EtOH): 232 nm (e=23,051), 247 nm (e=24,678).

¹HNMR (300 MHz, CD4OD) d: 7.14-7.37 (m, 12H), 7.06 (d, J=1.9 5 Hz, J=8.3 Hz, 1H), 6.86 (d, J=8.4 Hz, 1H), 6.65 (m, 1H), 6.40 (d, J=15.8 Hz, 1H), 5.92-6.05 (m, 2H), 4.97 (m, 2H), 4.51 (m, 1H), 3.73 (s, 3H), 3.66 (m, 2H), 2.80-3.15 (m, 4H), 2.40-2.72 (m, 3H), 1.40-1.70 (m, 3H), 1.09 (d, J=6.7 Hz, 3H), 0.77 (d, J=6.4 Hz, 3H), 0.68 (d, J=6.4 hz, 3H)

Mass(FD): 733.4 (M⁺+H).

Anal: Calcd for C41H49N2O8Cl (TFA salt): C, 67.17; H, 6.69; N, 3.82. Found: C, 68.04; H, 6.57, N, 3.47.

### EXAMPLE 9

### Synthesis of Compound (O)

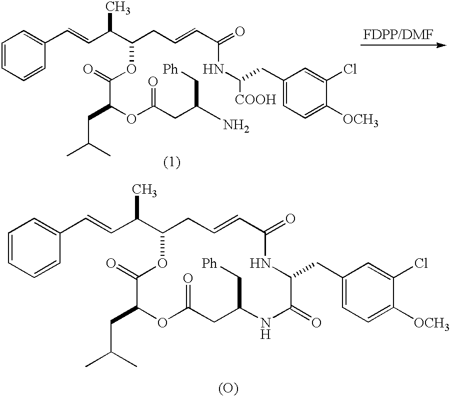

A sample of 0.25 g (0.295 mmol) of (1) was dissolved in 50 ml of dry DMF under argon atmosphere and to this was then added 0.147 g (0.384 mmol, 1.3 eq) of pentafluorophenyl-diphenylphosphinate (FDPP). The resulting reaction mixture was then let stirred at room temperature for 18 h. Another 30 mg of FDPP was added after 18 h due to some unreacted starting material. The reaction was completed after stirring at room temperature for another 4 h. DMF was then removed in vacuo and the residue was triturated with hexane to give a crude solid (300 mg). This solid was then flash chromatographed on SiO₂ (5% CH3OH/CHCl3) to give 0.183 g (86.7%, based on TFA salt as the starting material) of cyclized compound (O) as white solid.

IR (cm⁻¹): 3415, 3029, 2962, 1747, 1721, 1678, 1651, 1524, 1503, 1487, 1464, 1280, 1259, 1181, 1148, 1067, 1006, 969.

UV (95%EtOH): 248 nm.

¹HNMR (300 MHz, CDCl₃) d: 7.85 (d, J=9.2 Hz, 1H), 7.19-7.37 (m, 10H), 7.06 (dd, J=1.4 Hz, J=7.5 Hz, 1H), 7.01 (dd, J=1.5 Hz, J=8.4 Hz, 1H), 6.81 (d, J=8.5 Hz, 1H), 6.77 (m, 1H), 6.39 (d, J=15.8 Hz, 1H), 6.00 (dd, J=8.8 Hz, J=15.8 H, 1H), 5.75 (d, J=16.3 Hz, 1H), 5.45 (d, J=7.7 Hz, 1H), 5.10 (m, 1H), 4.80 (m, 2H), 4.69 (m, 1H), 4.32 (m, 1H), 3.87 (s, 3H), 3.12 (dd, J=6.5 Hz, J=14.3 Hz, 1H), 2.97 (m, 2H), 2.38-2.62 (m, 4H), 1.65 (m, 2H), 1.27 (m, 1H), 1.13 (d, J=6.9 Hz, 3H), 0.77 (d, J=6.3 Hz, 3H), 0.72 (d, J=6.3 Hz, 3H).

Mass(FAB): 715.3 (M⁺+H).

Anal: Calcd for C41H47N2O7C1: C, 68.85; H, 6.62; N, 3.92. Found: C, 68.89; H, 6.35, N, 4.00.

### EXAMPLE 10

### Synthesis of Epoxides (P) and (Q)

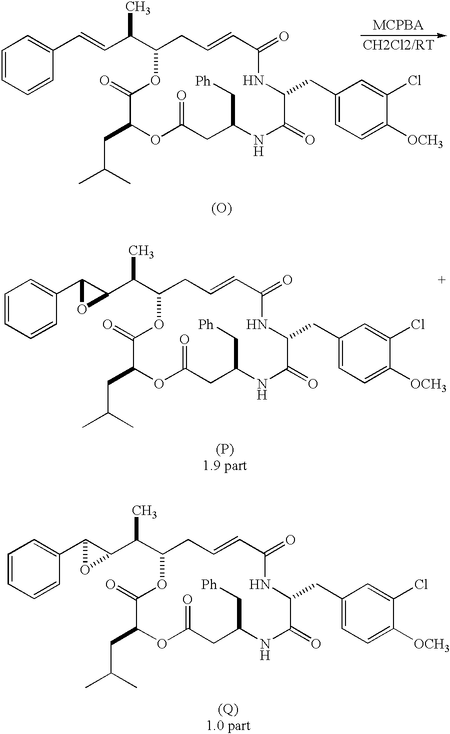

A sample of 120 mg (0.168 mmol) of compound (O) was dissolved in 6 ml of dry methylene chloride, to this was then added 70 mg (2.0 eq) of MCPBA at room temperature. This was then let stirred under argon for 18 h. The reaction mixture was checked by HPLC (2×4.6 mm×15 cm Novapak C-18 column, 75/25 CH₃CN:H2O, 1.0 ml/min, monitored at 254 nm) and it was found that ca 50% of starting material still remain after 18 h. Another 35 mg (1.0 eq) of MCPBA was added and reaction again monitored by HPLC. After stirring overnight (ca 41 h total) reaction, 20 mg of MCPBA was added (total: 125 mg, 3.6 eq) to the reaction mixture. The reaction was let stirred for another 6 h until HPLC showed only 1.3% of starting material remained unreacted. HPLC also indicated the two epoxides (P) and (Q) existed in a ratio of ca. 1.9:1.0. of the 5.5ml of the reaction mixture, 1.5 ml of the sample (ca 30% eq) was removed and used immediately for the chlorohydrin reaction for the next step (see next experimental). Of the remaining 70% (ca. 4.0 ml) reaction mixture, it was then diluted with methylene chloride (50 ml) and washed with 5%NaHCO₃ (20 ml×2) to remove the unreacted MCPBA and metachlorobenzoic acid. The organic layer was then dried over Na₂SO₄ and concentrated in vacuo to give 115 mg of pale yellow solid.

### EXAMPLE 11

### Synthesis of Chlorohydrin Compounds (R) and (S)

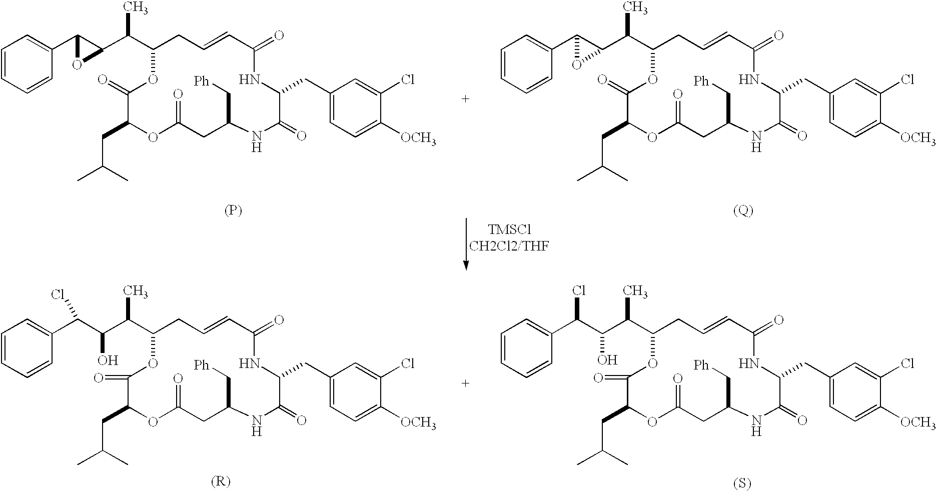

To the 1.5 ml methylene chloride solution obtained from the previous MCPBA reaction (30% eq, ca 30 mg of epoxides in theory, 0.04 mmol) was added another 1.0 ml of dry THF.

The reaction mixture was then kept under argon atmosphere and cooled to −60 C. To this was added 26 ul (22 mg, 5.0 eq) of trimethylchlorosilane. The reaction was then followed by TLC (5% CH30H/CHC13), the reaction was let stirred at −60 C. for 3 h before another 100 ul of TMSC1 was added. TLC showed the appearance of two more polar spots with ratio of ca 2:1. The reaction was then terminated after stirring at −40 C. for another 3 h (TLC still showed some unreacted material). The solvent was then removed in vacuo to give a white solid which was further purified by preparative reversed phase HPLC giving the two chlorohydrins (R) and (S).

### EXAMPLE 12

### Synthesis of Compound (A)

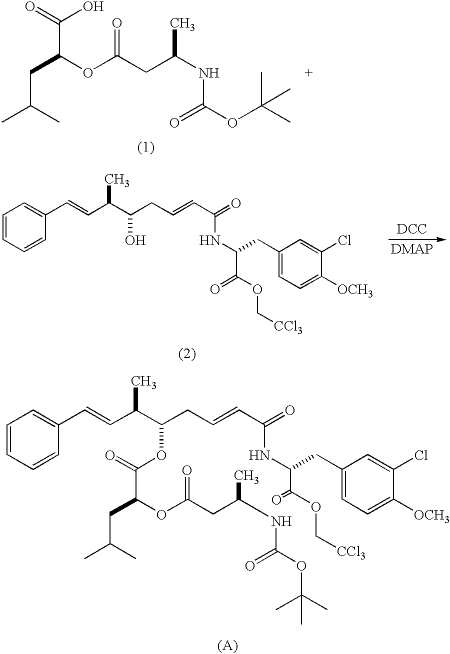

To a flame-dried 100 ml three-neck round bottom flask under argon was added 0.80 g (2.52 mmol, 1.5 eq) of (2S)-2-[3′(tert-Butoxycarbonyl)amino-2′-(R)-methylpropanoyloxy]-4-methylpentanoic acid (1) and 0.99 g (1.68 mmol) of compound (2) in 50 ml of dry methylene chloride at 0° C. To this solution was then added 50 mg of DMAP and 0.52 g (2.52 mmol, 1.50 eq) of dicyclohexylcarbodiimide and the resulting solution was let stirred at RT for 4 h. TLC showed the completion of the reaction after 4 h and the white precipitate was filtered through a short pad of celite and the filtrate was diluted with 500 ml of ether. The organic layer was then washed with 50 ml of 1.0 N HCl followed by 50 ml of 5% NaHCO3. The solvent was then removed in vacuo to give a crude solid which weight ca. 1.70 g. This crude solid was then flash chromatographed on SiO2 (5% EtOAc/methylene chloride) to give a total of 1.06 g (72%) of coupled product (A) as a foamed solid.

TLC: Rf=0.52 (1:1 EtOAc/hexane)

IR (cm⁻¹): 2964, 1743, 1713, 1677, 1642, 1504, 1367, 1281, 1259, 1170, 1127, 1066.

UV (CH3OH): 246 nm (e=21,047).

¹HNMR (300 MHz, DMSO-d6) d: 7.31 (m, 5H), 7.19 (d, J=2.0 Hz, 1H), 7.06 (dd, J=2.0 Hz, J=8.4 Hz, 1H), 6.82 (d, J=8.4 Hz, 1H), 6.65-6.78 (m, 2H), 6.38 (d, J=15.9 Hz, 1H), 6.00 (dd, J=8.7 Hz, J=15.9 Hz, 1H), 5.89 (d, J=15.6 Hz, 1H), 5.03 (m, 2H), 4.90 (dd, J=3.7 Hz, J=9.9 Hz, 1H), 4.79 (d, J=11.9 Hz, 1H), 4.69 (d, J=11.9 Hz, 1H), 4.00 (m, 1H), 3.85 (S, 3H), 3.14 (m, 2H), 2.52 (m, 6H), 1.50-1.75 (m, 3H), 1.41 (s, 9H), 1.18 (d, J=6.7 Hz, 3H), 1.10 (d, J=6.8 Hz, 3H), 0.84 (d, J=6.4 Hz, 3H), 0.79 (d, J=6.4 hz, 3H).

Mass(FD): 888.8 (M⁺).

Anal: Calcd for C42H54N2O10C14: C, 56.76; H, 6.12; N, 3.15. Found: C, 56.54; H, 6.16, N, 3.11.

### EXAMPLE 13

### Synthesis of Compound (B)

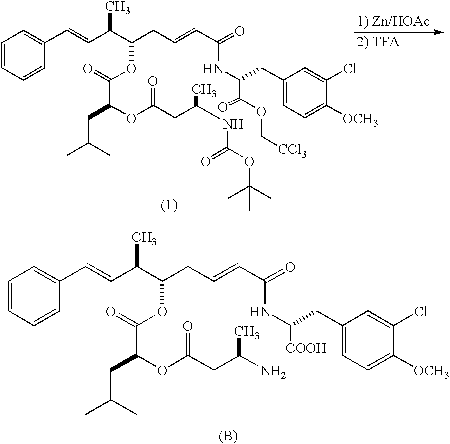

A sample of 0.98 g (1.12 mmol) of (1) in 40 ml of glacial acetic acid and to this was added 3.9 g of zinc dust. The mixture was then sonicated (room temperature) fro 45 min (TLC showed the completion of the starting material). The zinc dust was then filtered away through a short pad of celite and the filtrate was concentrated to give a white solid. This crude solid was then dissolved immediately in 50 ml of trifluoroacetic acid and let stirred at room temperature for 2 h. TFA was then removed in vacuo and the oily solid was triturated with ether/hexane to give a white solid (1.30 g). This crude solid was then flash chromatographed on SiO2 (10% CH3OH/CHCl3) to give a white solid (1.0 g). This solid was then dissolved in ca. 50 ml of distilled water and triturated for 1 h. The white solid was then collected and dried in vacuo at 50° C. to give 0.62 g (72% in two steps) of TFA salt of compound (B) as a white solid.

TLC: Rf=0.19 (10% CH3OH/CHCL3)

IR (cm⁻¹): 2961, 2935, 1741, 1674, 1621, 1503, 1442, 1393, 1281, 1258, 1202, 1144, 1127, 1066, 970.

UV (CH3OH): 230 nm (e=24,055), 247 nm (e=24,515).

¹HNMR(300 MHz, CD40D) d: 7.30 (m, 5H), 7.17 (d, J=2.1 Hz, 1H), 7.09 (dd, J=1.9 Hz, J=8.4 Hz, 1H), 6.90 (d, J=8.4 Hz, 1H), 6.65 (m, 1H), 6.40 (d, J=15.8 Hz, 1H), 6.02 (m, 2H), 4.93 (m, 2H), 4.53 (m, 1H), 3.79 (s, 3H), 3.85 (m, 1H), 3.57 (m, 1H), 3.14 (m, 2H), 2.90 (m, 2H), 2.62 (m, 4H), 1.42-1.70 (m, 3H), 1.27 (d, J=6.7 Hz, 3H), 1.10 (d, J=6.8 Hz, 3H), 0.77 (d, J=6.4 Hz, 3H), 0.70 (d, J=6.4 hz, 3H).

Mass(FD): 785.4 (M⁺).

Anal: Calcd for C38H48N2O10F3C1 (TFA salt): C, 58.12; H, 6.16; N, 3.57. Found: C, 57.92; H, 6.11, N, 3.91.

### EXAMPLE 14

### Synthesis of compound (C)

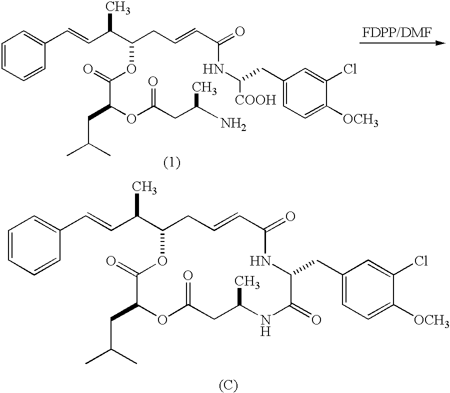

A sample of 0.30 g (0.39 mmol) of (1) was dissolved in 50 ml of dry DMF under argon atmosphere and to this was then added 0.19 g (0.51 mmol, 1.3 eq) of pentafluorophenyl-diphenylphosphinate (FDPP) in 8 ml of dry DMF. The resulting reaction mixture was then let stirred at room temperature for 5 h. DMF was then removed in vacuo and the residue was triturated with ether/hexane to give a crude solid. This solid was then flash chromatographed on SiO2 (5% CH30H/CHCl3) to give 0.18 g (72%) of cyclized compound (C) as white solid.

TLC: Rf=0.21 (10% CH3OH/CHCL3)

IR (cm⁻¹): 3394, 3290, 2960, 1744, 1728, 1676, 1659, 1539, 1521, 1503, 1442, 1204, 1173, 751.

UV (CH3OH): 247 nm (e=21,980).

¹HNMR(300 MHz, CDCl₃) d: 7.85 (d, J=9.6 Hz, 1H), 7.31 (m, 5H), 7.19 (d, J=2.0 Hz, 1H), 7.03 (dd, J=2.0 Hz, J=8.3 Hz, 1H), 6.83 (d, J=8.2 Hz, 1H), 6.79 (m, 1H), 6.38 (d, J=15.9 Hz, 1H), 6.00 (dd, J=8.8 Hz, J=15.3 H, 1H), 5.74 (d, J=14.7hz, 1H), 5.39 (d, J=8 Hz, 1H), 5.12 (m, 1H), 4.77 (m, 2H), 4.25 (m, 1H), 3.88 (s, 3H), 3.26 (dd, J=5.8 Hz, J=13.2 Hz, 1H), 2.94 (dd, J=5.2 Hz, J=14.3 Hz, 1H), 2.42-2.70 (m, 4H), 1.64 (m, 2H), 1.31 (m, 1H), 1.14 (d, J=4.4 Hz, 3H), 1.12 (d, J=4.4 Hz, 3H), 0.74 (d, J=2.9 Hz, 3H), 0.71 (d, J=2.9 Hz, 3H).

Mass (FAB): 639.4 (M⁺).

Anal: Calcd for C35H43N2O7Cl: C, 66.20; H, 6.94; N, 4.29. Found: C, 66.04; H, 6.82, N, 4.38.

### EXAMPLE 15

### The Synthesis of Epoxides (D) and (E)

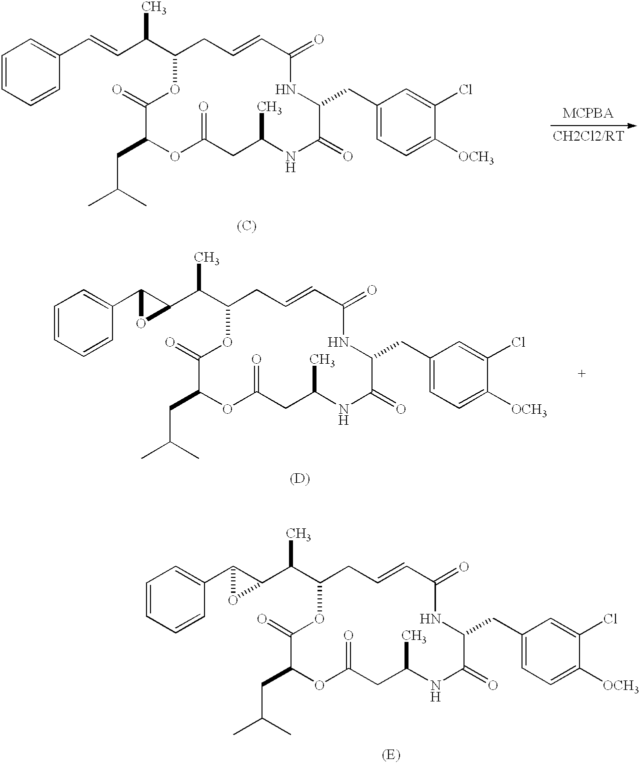

A sample of compound (C) was dissolved in dry methylene chloride, to this was then added (2.0 eq) of MCPBA at room temperature. This was then let stirred under argon for 18 h. The reaction mixture was checked by HPLC (2×4.6m×15 cm Novapak C-18 column, 75/25 CH3CN:H2O, 1.0 ml/min, monitored at 254 nm) and it was found that starting material still remained after 18 h. Another (1.0 eq) of MCPBA was added and reaction again monitored by HPLC. After stirring overnight MCPBA was added to the reaction mixture. The reaction was let stirred for another 6 h until HPLC showed only 1.3% of starting material remained unreacted. Of the 5.5ml of the reaction mixture, 1.5 ml of the sample (ca 30 % eq) was removed and used immediately for the chlorohydrin reaction for the next step (see next experimental). Of the remaining 70% (ca. 4.0 ml) reaction mixture, it was then diluted with methylene chloride (50 ml) and washed with 5%NaHCO3 (20 ml×2) to remove the unreacted MCPBA and metachlorobenzoic acid. The organic layer was then dried over Na2SO4 and concentrated in vacuo to give pale yellow solid which was purified and separated on a semiprep reversed phase C-18 HPLC column to give the two epoxides (D) and (E).

### EXAMPLE 16

### Synthesis of Chlorohydrin Compounds (F) and (G)

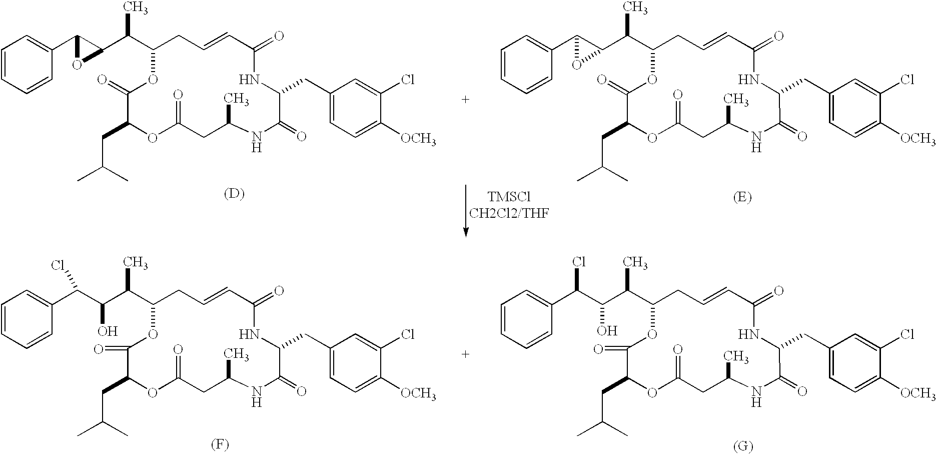

To the methylene chloride solution obtained from the previous MCPBA reaction was added another 1.0 ml of dry THF. The reaction mixture was then kept under argon atmosphere and cooled to −60 C. To this was added 5.0 eq of trimethyl-chlorosilane. The reaction was then followed by TLC (5% CH3OH/CHCl3), the reaction was let stirred at −60° C. for 3 h before another aliquot of TMSC1 was added. TLC showed the appearance of two more polar spots with ratio of ca 2:1. The reaction was then terminated after stirring at −40 C. for another 3 h (TLC still showed some unreacted material). The solvent was then removed in vacuo to give a white solid which was further purified by preparative reversed phase HPLC to give the two chlorohydrins (F) and (G).

### EXAMPLE 17

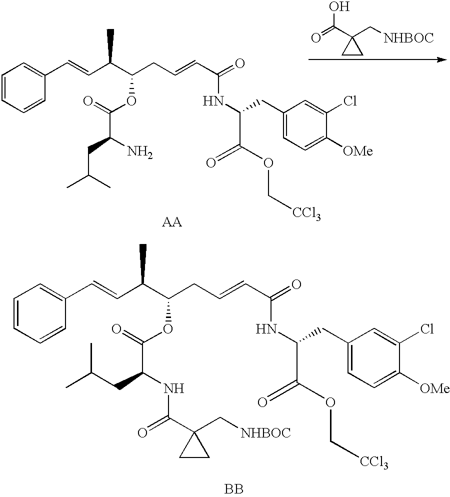

After flame drying, under nitrogen, a 100 mL 14/20 3-neck round bottom flask, 0.81 g (3.8 mmol) of N-tert-butoxycarbonyl 1-aminomethyl-1-cyclopropanecarboxylic acid was dissolved in 10 mL of anhydrous THF, followed by the addition of 0.81 g (3.8 mmol) of 1-ethyl-3-(3-dimethylaminopropyl)carbodiimide and 0.64 g (4.75 mmol) of 1-hydroxybenzotriazole. Next, 10 mL of anhydrous DMF were added and a solution resulted. To this solution was then added 1.35 g (1.65 mmol) of AA and 0.31 mL (2.85 mmol) of 4-methylmorpholine dissolved in 5 mL of anhydrous DMF. The reaction was stirred at room temperature for 2 hours. The volatiles were removed in vacuo, and the residue was dissolved in 50 mL of EtOAc and washed with 0.1 N HCl, brine, dried over Na₂SO₄, and removed in vacuo. This crude solid was purified on silica gel using flash chromatography, eluting with 20% EtOAc/Hex to give 1.14 g (77%) of BB as a white solid.

m.p.=73-75° C.

Mass (FD) M+1=900

### EXAMPLE 18

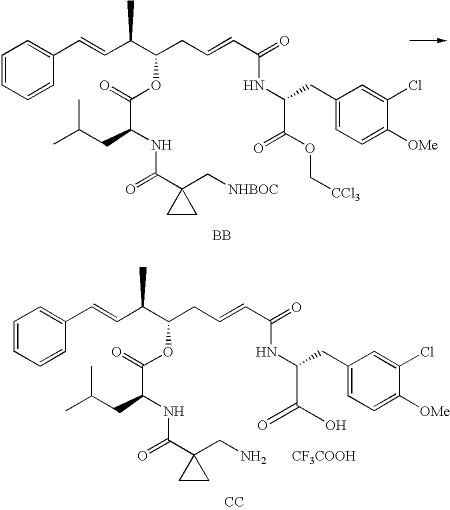

To a 250 mL round bottom flask were charged 1.1 g (1.22 mmol) of BB and 4.0 g of zinc dust in 40 mL of glacial acetic acid. The mixture was sonicated for 45 min and then stirred at room temperature for an additional 45 min. The reaction was filtered through celite, washed with fresh HOAc and MeCl₂, and the filtrate was removed in vacuo and pumped dry. This white solid was then dissolved in 40 mL of trifluoroacetic acid and stirred at room temperature for 2 hours. The TFA was removed in vacuo, and this crude residue was purified on silica gel using flash chromatography, eluting with 20% MeOH/CHCl₃ to give 0.77 g (81%) of CC as a white solid.

m.p.=131-134° C.

Mass (FD) M+=668

### EXAMPLE 19

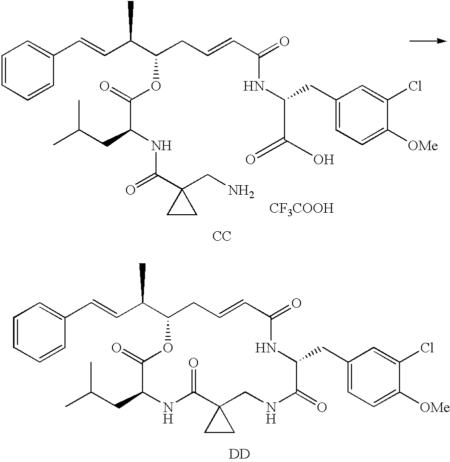

To a flame dried 250 mL 14/20 round bottom flask under nitrogen were charged 0.76 g (0.97 mmol) of CC and 1.02 mL (5.83 mmol) of anhydrous N,N-diisopropylethylamine in 125 mL of anhydrous DMF. Then 0.48 g (1.26 mmol) of pentafluorodiphenylphosphinate dissolved in 18 mL of anhydrous DMF were added dropwise to the stirring solution and the reaction was stirred for 4 hours. The DMF was removed in vacuo, and the residue was dissolved in CHCl₃ and washed with water, brine, dried over NaSO₄, and removed in vacuo. The crude residue was purified on silica gel using flash chromatography, eluting with 100% EtOAc to give 0.52 g (82%) of DD as a white solid.

m.p.=114-117° C.

Mass (FD) M+650

### EXAMPLE 20

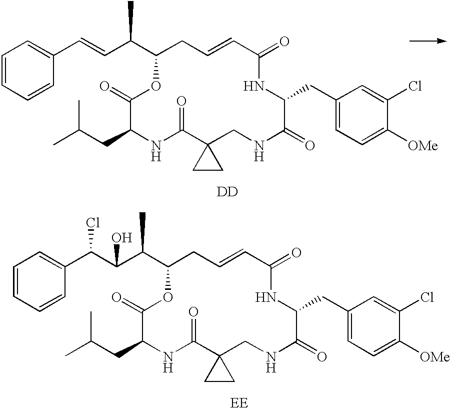

After flame drying a 15 mL 14/20 round bottom flask under nitrogen, 0.49 g (0.75 mmol) of DD was dissolved in 5 mL of anhydrous MeCl₂. Next, 0.14 g (0.79 mmol) of purified 3-chloroperbenzoic acid was added and the reaction was stirred at room temperature for 23 hours. The reaction was diluted with some additional MeCl₂, and washed with 10% Na₂S₂O₅, brine, 5% NaHCO₃, brine, dried over NaSO₄, and removed in vacuo to give 0.45 g (90%) of a crude white solid as a mixture of the α and β epoxides which was reacted directly without further purification into the next step. To a 50 ml 14/20 round bottom flask was dissolved 0.43 g (0.675 mmol) of the isolated epoxide mixture in 13 mL of anhydrous CHCl₃. The solution was cooled down in an ice bath, followed by the addition of 0.34 mL (2.7 mmol) of chlorotrimethylsilane. The ice bath was then removed, and the reaction was stirred at room temperature for 2.5 hours. The volatiles were removed in vacuo, and the crude residue was purified on silica gel using flash chromatography, eluting with 1% MeOH/EtOAc to give 0.16 g (34%) of the β-chlorohydrin EE as a white solid.

m.p.=159-162° C.

MS(FD) m/e 702 (M⁺)

### EXAMPLE 21

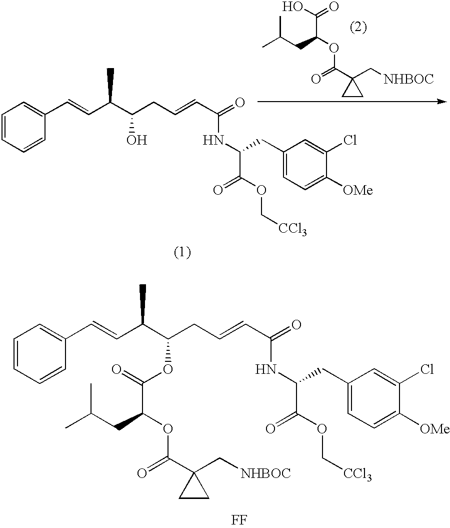

Beginning with 1.5 gm (2.55 mMol) (1) and 0.88 gm (2.68 mMol) (2), 1.7 gm (74%) compound FF was prepared as a white solid essentially as described in Example 7.

m.p.=56-59° C.

MS(FD): m/e=900

EA: Calculated for: C₄₃H₅₄N₂O₁₀Cl₄: C, 57.34; H, 6.04; N, 3.11. Found: C, 57.52; H, 6.13; N, 3.02.

### EXAMPLE 22

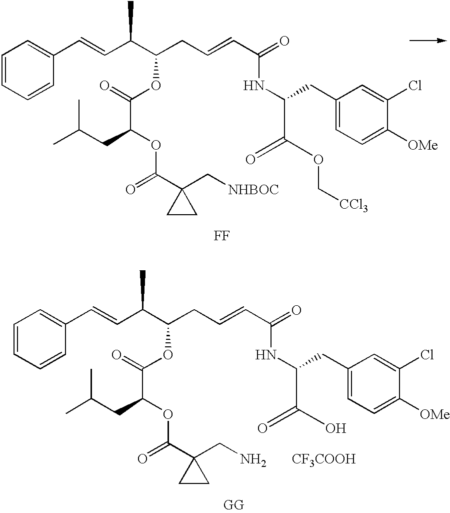

Beginning with 1.66 gm (1.84 mMol) FF, 1.1 gm (76%) GG was prepared as a tan solid essentially as described in Example 18.

m.p.=93-95° C.

MS(FD): m/e=669

HRMS: Calculated for: C₃₆H₄₅N₂O₈Cl: 669.2953. Found: 669.2943.

### EXAMPLE 23

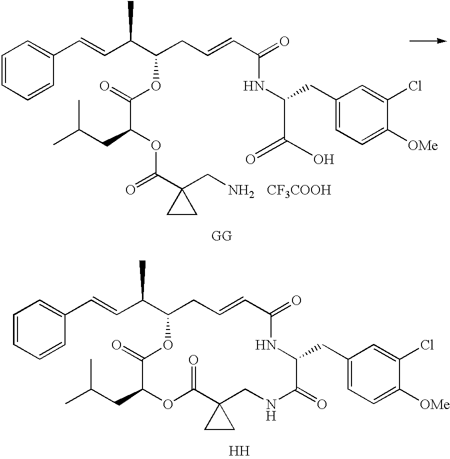

Beginning with 1.07 gm (1.37 mMol) GG, 0.73 gm (82%) HH was prepared as a white solid essentially as described in Example 19.

m.p.=123° C.

MS(FD): m/e=650

EA: Calculated for: C₃₆H₄₃N₂O₇Cl: C, 66.40; H, 6.66; N, 4.30. Found: C, 66.62; H, 6.69; N, 4.25.

### EXAMPLE 24

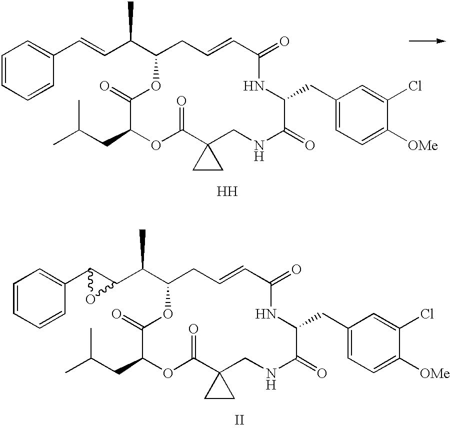

A solution of 0.68 gm (1.06 mMol) HH and 0.19 gm 3-chloroperbenzoic acid in 7 mL dichloromethane was stirred at room temperature. Additional portions of 3-chloroperbenzoic acid, 0.010 gm and 0.030 gm, were added at 23 hours and 26 hours respectively. Once all starting material was consumed the reaction mixture was diluted with 75 mL dichloromethane and the resulting solution washed sequentially with 10% aqueous sodium hydrosulfite (three times), water, 5% aqueous sodium bicarbonate (three times), and saturated aqueous sodium chloride. The remaining organic phase was dried over sodium sulfate and concentrated under reduced pressure to provide 0.65 gm crude II. A 0.050 gm portion of this mixture of epoxides was subjected to HPLC chromatography to provide:

β-epoxide

0.015 gm

m.p.=121-123° C.

HRMS: Calculated for: C₃₆H₄₃N₂O₈Cl: 667.2786. Found: 667.2783.

α-epoxide

0.008 gm

m.p.=108-110° C.

HRMS: Calculated for: C₃₆H₄₃N₂O₈Cl: 667.2786. Found: 667.2789.

### EXAMPLE 25

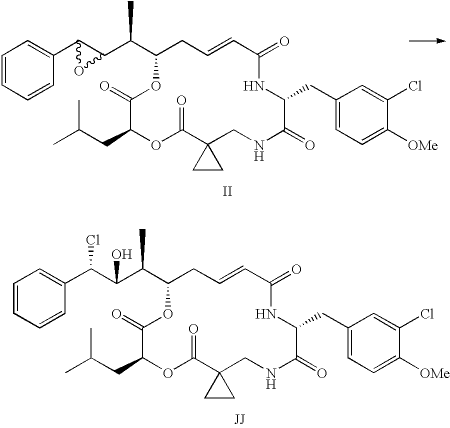

A solution of 0.35 gm (0.52 mMol) II in 10 mL chloroform was cooled in an ice bath and then 0.27 (2.1 mMol) trimethylsilyl chloride were added. The reaction mixture was stirred for 2 hours in the ice bath and then the reaction mixture was concentrated under reduced pressure. The residual solid was subjected to silica gel chromatography, eluting with 1:1 dichloromethane:ethyl acetate. Fractions containing product were combined and concentrated under reduced pressure to provide 0.16 gm JJ as a white solid.

m.p.=137° C.

MS(FD): m/e=702

EA: Calculated for: C₃₆H₄₄N₂O₈Cl₂: C, 61.45; H, 6.30; N, 3.98. Found: C, 61.32; H, 6.38; N, 4.27.

### EXAMPLE 26

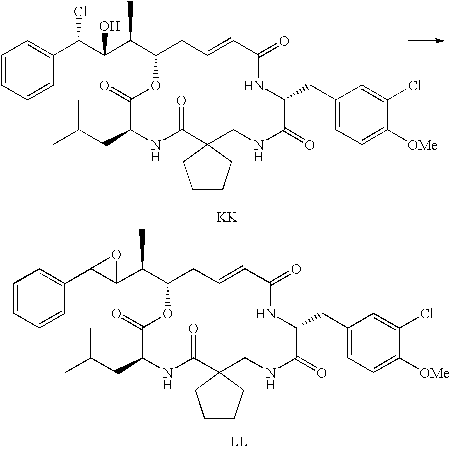

Beginning with N-[tert-butoxycarbonyl] O-[1-carboxy-3-methylbut-1-yl] 1-aminomethyl-cyclopentanecarboxylate, chlorohydrin KK was prepared essentially as described in Examples 7-11. A mixture of 0.021 gm KK and 0.008 gm (2 equivalents) potassium carbonate in 100 μL acetonitrile and 50 μL water was stirred at room temperature for 4 hours. The reaction mixture was diluted with 3 mL methyl tert-butyl ether and 1 mL of water and the phases were separated. The aqueous phase was extracted with 3×3 mL methyl tert-butyl ether and the combined organic phases are washed with 2 mL water, dried over sodium sulfate and concentrated under reduced pressure. A solution of this residue in methyl tert-butyl ether was filtered through silica gel and the filtrate concentrated under reduced pressure to provide 0.014 gm of LL as a white foam.

MS(ES): m/e=695 (M⁺)

### EXAMPLE 27

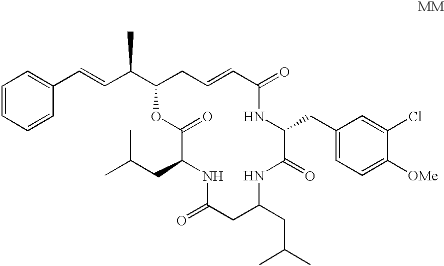

Beginning with (2S) -2-[3′(tert-Butoxycarbonyl)amino-5′-methylhexanoyloxy]-4-methylpentanoic acid, the desired compound was prepared essentially as described in Examples 7-9.

m.p.=258-260° C.

HRMS: Calculated for C₃₈H₄₉N₂O₇Cl: 681.3307. Found: 681.3316.

### EXAMPLE 28

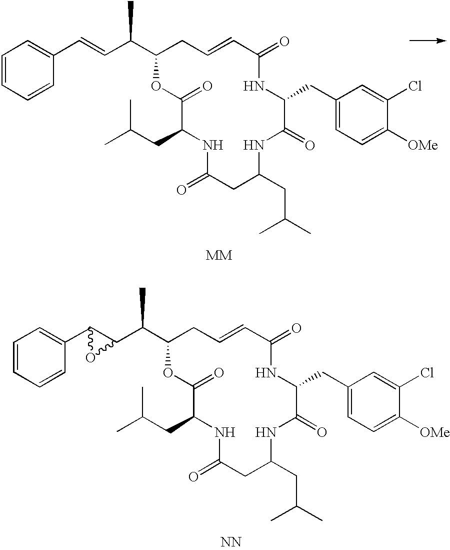

Beginning with 0.040 gm (.0587 mMol) MM, 0.020 gm NN was prepared essentially as described in Example 24. This material was subjected to chromatography to provide:

β-epoxide

0.006 gm

m.p.=255-257° C.

HRMS: Calculated for: C₃₈H₄₉N₂O₈Cl: 697.3256. Found: 697.3263.

α-epoxide

0.002 gm

m.p.=193-195° C.

### EXAMPLE 29

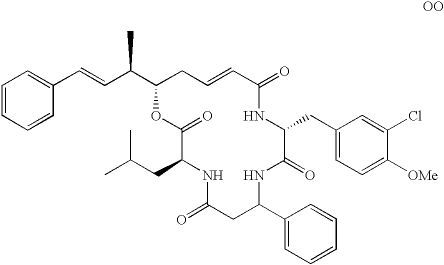

Beginning with (2S)-2-[3′(tert-Butoxycarbonyl)amino-3′-phenylpropanoyloxy]-4-methylpentanoic acid, the desired compound was prepared essentially as described in Examples 7-9.

m.p.=284-286° C.

EA: Calculated for: C₄₀H₄₅N₂O₇Cl: C, 68.51; H, 6.47; N, 3.99. Found: C, 68.73; H, 6.51; N, 3.91.

### EXAMPLE 30

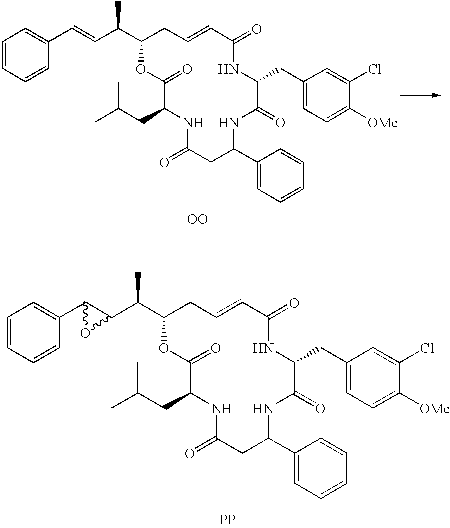

Beginning with 0.144 gm (.205 mMol) OO, 0.14 gm PP was prepared essentially as described in Example 24. This material was subjected to chromatography to provide:

β-epoxide

HRMS: Calculated for: C₄₀H₄₅N₂O₈Cl: 717.2943. Found: 717.2933.

α-epoxide

HRMS: Calculated for: C₄₀H₄₅N₂O₈Cl: 717.2943. Found: 717.2940.

We claim:

1. A compound of Formula I 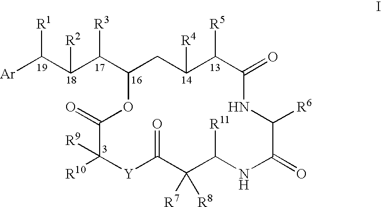
wherein
Ar is phenyl with a substituent selected from the group consisting of hydrogen, hydroxy, lower alkoxy, halogen, and lower alkyl;
R¹ is halogen and R² is hydroxy, or R¹ and R² may be taken together to form an epoxide ring or a second bond between C₁₈ and C₁₉;
R³ is a lower alkyl group;
R⁴ and R⁵ are hydrogen, or R⁴ and R⁵ may be taken together to form a second bond between C₁₃ and C₁₄;
R⁶ is selected from the group consisting of benzyl, hydroxybenzyl, alkoxybenzyl, halohydroxybenzyl, dihalohydroxybenzyl, haloalkoxybenzyl, and dihaloalkoxybenzyl;
R⁷ and R⁸ are each hydrogen and Rll is selected from the group consisting of hydroxy, lower alkyl, unsubstituted phenyl, substituted phenyl, unsubstituted benzyl, and substituted benzyl, or R⁷ and R⁸ form a spiro group and R¹¹ is hydrogen;
R⁹ is hydrogen or a lower alkyl group;
R¹⁰ is hydrogen;
Y is selected from the group consisting of O and NH;
or a pharmaceutically acceptable salt or solvate thereof.

2. A compound of claim 1 where Ar is phenyl.

3. A compound of claim 2 where R⁶ is substituted benzyl wherein one substituent is a halogen and one is an OR¹² wherein R¹² is lower alkyl.

4. A compound of claim 3 where R⁶ is 3-chloro-4-methyoxybenzyl.

5. A compound of claim 4 where R⁷ and R⁸ are each hydrogen.

6. A compound of claim 5 where R¹¹ is methyl.

7. A compound of claim 4 where R⁷ and R⁸ form a spiro group.

8. A compound of claim 7 where R⁷ and R⁸ form a cyclopropyl.

9. A pharmaceutical formulation comprising a compound of Formula I 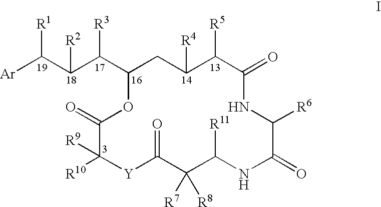
wherein
Ar is phenyl with a substituent selected from the group consisting of hydrogen, hydroxy, lower alkoxy, halogen, and lower alkyl;
R¹ is halogen and R² is hydroxy, or R¹ and R² may be taken together to form an epoxide ring or a second bond between C₁₈ and C₁₉;
R³ is a lower alkyl group;
R⁴ and R⁵ are hydrogen, or R⁴ and R⁵ may be taken together to form a second bond between C₁₃ and C₁₄;
R⁶ is selected from the group consisting of benzyl, hydroxybenzyl, alkoxybenzyl, halohydroxybenzyl, dihalohydroxybenzyl, haloalkoxybenzyl, and dihaloalkoxybenzyl;
R⁷ and R⁸ are each hydrogen and R¹¹ is selected from the group consisting of hydroxy, lower alkyl, unsubstituted phenyl, substituted phenyl, unsubstituted benzyl, and substituted benzyl, or R⁷ and R⁸ form a spiro group and R¹¹ is hydrogen;
R⁹ is hydrogen or a lower alkyl group;
R¹⁰ is hydrogen;
Y is selected from the group consisting of O and NH;
or a pharmaceutically acceptable salt or solvate thereof in combination with at least one pharmaceutically acceptable diluent or carrier therefore.

10. A method for treating a neoplasm in a mammal comprising administering to a mammal in need of said treatment an effective amount of a compound of Formula I: 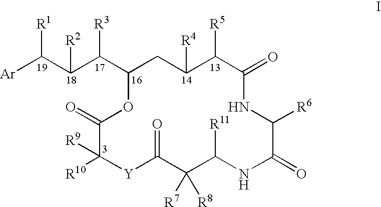
wherein
Ar is phenyl with a substituent selected from the group consisting of hydrogen, hydroxy, lower alkoxy, halogen, and lower alkyl;
R¹ is halogen and R² is hydroxy, or R¹ and R² may be taken together to form an epoxide ring or a second bond between C₁₈ and C₁₉;
R³ is a lower alkyl group;
R⁴ and R⁵ are hydrogen, or R⁴ and R⁵ may be taken together to form a second bond between C₁₃ and C₁₄;
R⁶ is selected from the group consisting of benzyl, hydroxybenzyl, alkoxybenzyl, halohydroxybenzyl, dihalohydroxybenzyl, haloalkoxybenzyl, and dihaloalkoxybenzyl;
R⁷ and R⁸ are each hydrogen and R¹¹ is selected from the group consisting of hydroxy, lower alkyl, unsubstituted phenyl, substituted phenyl, unsubstituted benzyl, and substituted benzyl, or R⁷ and R⁸ form a Spiro group and R¹¹ is hydrogen;
R⁹ is hydrogen or a lower alkyl group;
R¹⁰ is hydrogen;
Y is selected from the group consisting of O and NH;
or a pharmaceutically acceptable salt or solvate thereof.
